SEQUENCE LISTING (8 sequences)

SEQ ID NO: 1 - protein, 5 aa, Artificial Sequence
  Synthetic Peptide
  MISC_FEATURE (1)..(5): Cyclo
  MOD_RES (1)..(1): MeVal
  MOD_RES (5)..(5): D-Phe
XRGDX

SEQ ID NO: 2 - protein, 9 aa, Artificial Sequence
  Synthetic Peptide
CFCDGRCDC

SEQ ID NO: 3 - protein, 5 aa, Artificial Sequence
  Synthetic Peptide
  MISC_FEATURE (1)..(5): Cyclo
  MOD_RES (4)..(4): D-Tyr
RGDXK

SEQ ID NO: 4 - protein, 5 aa, Artificial Sequence
  Synthetic Peptide
  MISC_FEATURE (1)..(5): Cyclo
  MOD_RES (4)..(4): D-Phe
RGDXC

SEQ ID NO: 5 - protein, 5 aa, Artificial Sequence
  Synthetic Peptide
  MISC_FEATURE (1)..(5): Cyclo
  MOD_RES (4)..(4): D-Phe
RGDXK

SEQ ID NO: 6 - protein, 7 aa, Artificial Sequence
  Synthetic Peptide
GRGDSPC

SEQ ID NO: 7 - protein, 16 aa, Artificial Sequence
  Synthetic Peptide
GCRDVPMSMRGGDRCG

SEQ ID NO: 8 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

(19) United States
(12) Patent Application Publication
(10) Pub. No.: US 2022/0033804 A1
(43) Pub. Date: Feb. 3, 2022

(54) Methods and Compositions for Enhancement of Stem Cell-based Immunomodulation and Tissue Repair

(71) Applicants: Georgia Tech Research Corporation, Atlanta, GA (US); The Regents of the University of Michigan, Ann Arbor, MI (US); Bolder BioTechnology, Inc., Boulder, CO (US)

(72) Inventors: Andres J. Garcia, Atlanta, GA (US); George N. Cox, Louisville, CO (US); Jose Garcia, Atlanta, GA (US); Asma Nusrat, Ann Arbor, MI (US); Miguel Angel Quiros Quesada, Ann Arbor, MI (US)

(21) Appl. No.: 17/390,190

(22) Filed: Jul. 30, 2021

Related U.S. Application Data
(60) Provisional application No. 63/058,743, filed on Jul. 30, 2020.

(51) Int. Cl. C12N 15/10

(52) U.S. Cl. CPC C12N 15/1044; C12N 15/1037

(57) ABSTRACT

Provided herein are methods and compositions for enhancement of stem-cell based immunomodulation and promotion of tissue repair.

### CROSS-REFERENCE TO RELATED APPLICATIONS

This application claims the benefit of priority under 35 U.S.C. § 119(e) to U.S. Provisional Patent Application No. 63/058,743, filed Jul. 30, 2020. The entire disclosure of U.S. Provisional Patent Application No. 63/058,743 is incorporated herein by reference.

### GOVERNMENT SUPPORT

This invention was made with government support under grant numbers R01 AR062368 and R01 DK055679 awarded by the National Institutes of Health. The government has certain rights in the invention.

### SEQUENCE LISTING

This application contains a Sequence Listing submitted electronically as a text file by EFS-Web. The text file, named “4152-22_Sequence_Listing_ST25”, has a size in bytes of 4000 bytes, and was recorded on Jul. 30, 2021. The information contained in the text file is incorporated herein by reference in its entirety pursuant to 37 CFR § 1.52(e)(5).

### BACKGROUND OF THE INVENTION

Human mesenchymal stem cells (hMSCs) are multipotent stromal cells that, in addition to having the ability to differentiate into cell types that produce distinct tissues (e.g., bone, cartilage and fat), exhibit potent immunomodulatory activities and are being evaluated in a myriad of clinical trials for treating autoimmune and chronic inflammatory diseases (Bruder S P, et al. Mesenchymal stem cells in bone development, bone repair, and skeletal regeneration therapy. J Cell Biochem. 1994; 56:283-94; Caplan AI. Adult mesenchymal stem cells for tissue engineering versus regenerative medicine. J Cell Physiol. 2007; 213:341-7; Aggarwal S, Pittenger M F. Human mesenchymal stem cells modulate allogeneic immune cell responses. Blood. 2005; 105:1815-22; Wang Y, et al. Plasticity of mesenchymal stem cells in immunomodulation: pathological and therapeutic implications. Nat Immunol. 2014; 15:1009-16; Gao F, et al. Mesenchymal stem cells and immunomodulation: current status and future prospects. Cell Death Dis. 2016; 7:e2062). Co-culturing hMSCs with activated T-cells or monocytes leads to reduced proliferation of T-cells and inhibition of monocyte-derived dendritic cell differentiation, respectively (Spaggiari G M, et al. MSCs inhibit monocyte-derived DC maturation and function by selectively interfering with the generation of immature DCs: central role of MSC-derived prostaglandin E2. Blood. 2009; 113:6576-83; Meisel R, et al. Human bone marrow stromal cells inhibit allogeneic T-cell responses by indoleamine 2,3-dioxygenase-mediated tryptophan degradation. Blood. 2004; 103:4619-21). hMSCs also have powerful inhibitory effects on other immune cell types ranging from natural killer cells to B-cells (Spaggiari G M, et al. Mesenchymal stem cells inhibit natural killer-cell proliferation, cytotoxicity, and cytokine production: role of indoleamine 2,3-dioxygenase and prostaglandin E2. Blood. 2008; 111:1327-33; Corcione A, et al. Human mesenchymal stem cells modulate B-cell functions. Blood. 2006; 107:367-72). Importantly, hMSC delivery ameliorates the effects of diverse autoimmune diseases in pre-clinical models of graft-vs-host disease (GvHD), colitis, and autoimmune encephalomyelitis (Auletta J J, et al. Human mesenchymal stromal cells attenuate graft-versus-host disease and maintain graft-versus-leukemia activity following experimental allogeneic bone marrow transplantation. Stem Cells. 2015; 33:601-14; Wang X, et al. Human ESC-derived MSCs outperform bone marrow MSCs in the treatment of an EAE model of multiple sclerosis. Stem Cell Reports. 2014; 3:115-30; Gonzalez M A, et al. Adipose-derived mesenchymal stem cells alleviate experimental colitis by inhibiting inflammatory and autoimmune responses. Gastroenterology. 2009; 136:978-89; Yanez R, et al. Adipose tissue-derived mesenchymal stem cells have in vivo immunosuppressive properties applicable for the control of the graft-versus-host disease. Stem Cells. 2006; 24:2582-91). Based on promising results in pre-clinical models, hMSCs have been evaluated in clinical trials for treating Crohn's disease as well as steroid-refractory acute GvHD, but with varying levels of success. In a phase II clinical study of refractory Crohn's disease, although the overall disease score was significantly reduced with administration of hMSCs, patient improvement was only noted in 7 of 15 patients (Forbes G M, et al. A phase 2 study of allogeneic mesenchymal stromal cells for luminal Crohn's disease refractory to biologic therapy. Clin Gastroenterol Hepatol. 2014; 12:64-71). Le Blanc and colleagues found that out of 55 patients having severe acute GvHD that received an infusion of hMSCs, 30 (55%) had a complete response while the other 25 had either a partial or no response to hMSC therapy (Le Blanc K, et al. Mesenchymal stem cells for treatment of steroid-resistant, severe, acute graft-versus-host disease: a phase II study. Lancet. 2008; 371:1579-86). Overall, these studies support the notion that hMSC therapy ameliorates autoimmune diseases, but the effect is only seen in approximately half of patients, leaving vast room for improvement (Resnick I B, et al. Treatment of severe steroid resistant acute GVHD with mesenchymal stromal cells (MSC). Am J Blood Res. 2013; 3:225-38; Panes J, et al. Expanded allogeneic adipose-derived mesenchymal stem cells (Cx601) for complex perianal fistulas in Crohn's disease: a phase 3 randomised, double-blind controlled trial. Lancet. 2016; 388:1281-90).

In order to fully elicit their immunomodulatory effects, hMSCs must be activated with pro-inflammatory stimuli, specifically interferon-gamma (IFN-γ), in a process termed ‘licensing’ (Krampera M, et al. Role for interferon-gamma in the immunomodulatory activity of human bone marrow mesenchymal stem cells. Stem Cells. 2006; 24:386-98; Lee M W, et al. Strategies to improve the immunosuppressive properties of human mesenchymal stem cells. Stem Cell Res Ther. 2015; 6:179). Either co-culturing hMSCs with IFN-γ-deficient immune cells or using antibodies to neutralize IFN-γ results in loss of hMSC immunomodulatory actions (Polchert D, et al. IFN-gamma activation of mesenchymal stem cells for treatment and prevention of graft versus host disease. Eur J Immunol. 2008; 38:1745-55; Liang C, et al. Interferon-gamma mediates the immunosuppression of bone marrow mesenchymal stem cells on T-lymphocytes in vitro. Hematology. 2018; 23:44-9). Once licensed with IFN-γ, hMSCs elicit their immunomodulatory effects by the upregulation of immunoactive factors including indoleamine 2,3-dixygenase (IDO), programmed death ligand-1 (PD-L1), prostaglandin E2 (PGE2), CCL8, CXCL9 and CXCL10 among many others (Jin P, et al. Interferon-gamma and tumor necrosis factor-alpha polarize bone marrow stromal cells uniformly to a Th1 phenotype. Sci Rep. 2016; 6:26345; Bernardo M E, Fibbe W E. Mesenchymal stromal cells: sensors and switchers of inflammation. Cell Stem Cell. 2013; 13:392-402). Importantly, the timing and duration of licensing are crucial, and licensing hMSCs prior to co-culture or use in vivo enhances their immunomodulatory capabilities (Shi Y, et al. How mesenchymal stem cells interact with tissue immune responses. Trends Immunol. 2012; 33:136-43). Licensing hMSCs with IFN-γ prior to co-culture with activated T-cells results in both inhibited T-cell proliferation and T-cell effector functions, whereas hMSCs that were not licensed prior to co-culture only inhibited T-cell proliferation (Chinnadurai R, et al. IDO-independent suppression of T cell effector function by IFN-gamma-licensed human mesenchymal stromal cells. J Immunol. 2014; 192:1491-501). Furthermore, licensing hMSCs prior to infusion into mice with GvHD results in enhanced hMSC-based suppression of GvHD compared to that of control un-licensed hMSCs (Polchert D, et al. IFN-gamma activation of mesenchymal stem cells for treatment and prevention of graft versus host disease. Eur J Immunol. 2008; 38:1745-55). Duijvestein et al also showed that delivering pre-licensed hMSCs significantly reduced the severity of experimental colitis in mice compared to un-licensed hMSCs (Duijvestein M, et al. Pretreatment with interferon-gamma enhances the therapeutic activity of mesenchymal stromal cells in animal models of colitis. Stem Cells. 2011; 29:1549-58).

Although ex vivo licensing of hMSCs is therapeutically effective, significant technical, regulatory, and economic issues limit the translational potential of this cell processing approach. Ex vivo manipulation, including extraction and isolation of hMSCs, plating onto culture supports, extended culturing conditions, and harvesting the licensed hMSCs, requires an efficient manufacturing process that complies with GMP and regulatory standards (Heathman T R, et al. The translation of cell-based therapies: clinical landscape and manufacturing challenges. Regen Med. 2015; 10:49-64; Tarnowski J, et al. Delivering advanced therapies: the big pharma approach. Gene Ther. 2017; 24:593-8). Furthermore, the increased cost necessary with manual or even automated processing presents a major burden that has contributed to the insolvency of many companies offering cell therapies (Dodson B P, Levine A D. Challenges in the translation and commercialization of cell therapies. BMC Biotechnol. 2015; 15:70). Therefore, generating a solution that bypasses the need for such processing would enhance the translatability and efficacy of this stem cell therapy.

Engineered biomaterials offer a potential solution for the need of ex vivo manipulation through scaffolds that provide necessary cues to encapsulated cells. Whereas biomaterials have been engineered to deliver factors that promote tissue healing and vascularization (Veith A P, et al. Therapeutic strategies for enhancing angiogenesis in wound healing. Adv Drug Deliv Rev. 2018; 50169-409X(18)30246; Annabi N, et al. 25th anniversary article: Rational design and applications of hydrogels in regenerative medicine. Adv Mater. 2014; 26:85-123), relatively little research has been done in engineering a scaffold to license and enhance the immunomodulatory activities of encapsulated hMSCs. Thus, there is a need in the art to establish a biomaterial-based strategy to enhance the immunomodulatory activities of hMSCs.

### BRIEF DESCRIPTION OF THE DRAWINGS

FIGS. 1A-1C: Tethering of cys-IFN-γ onto PEG-4MAL hydrogels and degradation-dependent release. (FIG. 1A) Schematic representing cytokine functionalization with adhesive ligand, hMSC and protease-degradable cross-linker incorporation. (FIG. 1B) Protein gel electrophoresis for cys-IFN-γ reacted with PEG-4MAL. Lane 1) protein ladder, lane 2) cys-IFN-γ reacted with PEG-4-MAL, lane 3) cys-IFN-γ. (FIG. 1C) Cys-IFN-γ release kinetics as measured by ELISA. All groups were incubated in PBS until 4 days at which point collagenase (50 μg/mL) was added to the respective group. N=5. Error bars±SEM.

FIGS. 2A-2I hMSCs on tissue culture plastic exhibit significant changes in marker expression and secreteome when incubated with IFN-γ compared to hMSCs without IFN-γ. FIG. 2A) Schematic of experimental outline. hMSCs were incubated with either cys-IFN-γ+PEG-4MAL, cys-IFN-γ, native IFN-γ, PEG-4MAL or no treatment. Following 4 days, hMSCs were stained for (FIG. 2B) IDO and (FIG. 2C) PD-L1 and subjected to flow cytometry. Conditioned media was analyzed for concentrations of various proteins including (FIG. 2D) IL-6, (FIG. 2E) CXCL10, (FIG. 2F) MCP-1, (FIG. 2G) VEGF, (FIG. 2H) CCL8 and (FIG. 21) M-CSF. Dotted lines signify limit of detection for specific protein. N=6. Error bars±SEM. One-way ANOVA **** p<0.0001.

FIGS. 3A-3I Licensing of hMSCs encapsulated in cys-IFN-γ-tethered hydrogels. (FIG. 3A) Schematic of experimental outline. hMSCs were encapsulated within hydrogels of different conditions and immunomodulatory properties analyzed. (FIG. 3B) hMSCs were encapsulated in 6% PEG wt %, 20 μL hydrogels with differing doses of cys-IFN-γ. Following 4 days of culture, cells were subjected to flow cytometric analysis for IDO expression. Dotted line indicates level of IDO expression of 0 ng dose. N=3-5. δ p<0.0001 vs all conditions tested except 0 ng dose. #p<0.05 vs 32 ng and 80 ng doses, p<0.001 vs 200 ng dose, p<0.0001 vs 0, 5, and 500 ng dose. $ p<0.001 vs 500 ng dose, p<0.0001 vs 0 ng dose. @ p<0.05 vs 500 ng dose, p<0.0001 vs 0 ng dose. † p<0.0001 vs 0 ng dose. (FIGS. 3C and 3D respectively) IDO and PD-L1 expression of hMSCs in hydrogels with cys-IFN-γ and IFN-γ. Following 4 days of culture, hMSCs in hydrogels with either cys-IFN-γ, IFN-γ or no IFN-γ were stained for (FIG. 3C) IDO and (FIG. 3D) PD-L1 and subjected to flow cytometric analysis. N=6. (FIGS. 3E-3I) Cytokine analysis of conditioned media. Conditioned media of hMSCs encapsulated in hydrogels with either cys-IFN-γ, IFN-γ or no IFN-γ was analyzed for (FIG. 3E) MCP-1, (FIG. 3F) M-CSF, (FIG. 3G) CXCL9, (FIG. 3H) CXCL10, (FIG. 3I) CCL8. N=6.Error bars±SEM. One-way ANOVA * p<0.05, ** p<0.01, *** p<0.001, **** p<0.0001.

FIGS. 4A-4L hMSCs encapsulated in cys-IFN-γ hydrogels modulate immune cells. (FIG. 4A) Schematic of experimental outline. hMSCs were encapsulated within hydrogels of different conditions and the effect on T-cells or monocytes analyzed. (FIGS. 4B-4H) hMSCs encapsulated within cys-IFN-γ hydrogels significantly reduce activated CD4+ T-cell proliferation. (FIGS. 4B-4G) Representative images of fluorescence microscopy images of proliferating T-cells stained for EdU, scale bar 100 μm. (FIG. 4H) Untreated or pre-licensed hMSCs were encapsulated within cys-IFN-γ, IFN-γ or no IFN-γ hydrogels and co-cultured with activated CD4+ T-cells for 4 days. T-cell proliferation was assessed via EdU incorporation. Graph shows samples from two independent experiments. N=5-8 separate wells with quantification of >100 T-cells per well. (FIG. 4I) Quantification of proliferating T-cells with IDO inhibitor. N=6-7 separate wells with quantification of >100 T-cells per well. (FIGS. 4J-4L) hMSCs in cys-IFN-γ hydrogels inhibit dendritic cell differentiation. (FIG. 4J) Percentage of dendritic cells in monocyte culture after 7 days differentiation as defined by CD1a+/CD14− by means of FMO controls. Median fluorescence intensity for markers (FIG. 4K) CD80 and (FIG. 4L) CD86. N=3-4 separate wells with 20,000 cells analyzed per well. Error bars±SEM. One-way ANOVA * p<0.05, ** p<0.01, *** p<0.001, **** p<0.0001.

FIGS. 5A-5E hMSCs in cys-IFN-γ hydrogels repair colonic wounds in immunocompetent mice. (FIG. 5A) Quantification of colonic wound closure at day 5 post-injury. (FIGS. 5B-5E) H&E staining of colonic wounds at day 5 post-injury. Hashed line delineates tissue border to indicate wound. Arrow points to crypts reforming within repaired tissue. N=5-9 mice. Error bars±SEM. One-way ANOVA ** p<0.01, *** p<0.001.

FIG. 6 Western blot for Cys-IFN-γ reacted with either PBS (lane 2) or PEG-4MAL (lane 3). IFN-γ was reacted with either PBS (lane 4) or PEG-4MAL (lane 5). Lane 1 is the protein ladder standard.

FIG. 7 Flow cytometry scattergrams and histograms for hMSCs incubated with cys-IFN-γ+PEG-4MAL, cys-IFN-γ, native IFN-γ, PEG-4MAL or no treatment. Raw data was gated to exclude cell debris (gate 1), followed by gating for IDO+ and DO− cells.

FIG. 8 Flow cytometry scattergrams and histograms for hMSCs incubated with cys-IFN-γ+PEG-4MAL, cys-IFN-γ, native IFN-γ, PEG-4MAL or no treatment. Raw data was gated to exclude cell debris (gate 1), followed by gating for IDO+ and DO− cells.

FIG. 9 hMSCs were encapsulated in hydrogels with differing cys-IFN-γ doses. Conditioned media at select doses was tested for kyneurenine concentration to verify correlation of IDO expression with IDO activity. Dotted line signifies concentration of kyneurenine in hMSC media. N=4-5. Error bars±SEM. One-way ANOVA *** p<0.001 vs 0 and 12.5 ng doses, **** p<0.0001 vs 0 and 12.5 ng doses.

FIG. 10 hMSCs were encapsulated within 20 μL hydrogels of varying PEG weight percentages and 500 ng cys-IFN-γ. After 4 days in culture, hMSCs were stained for IDO and PD-L1 and subject to flow cytometric analysis. N=5. Error bars±SEM, * p<0.05, ** p<0.01, *** p<0.001.

FIG. 11 hMSCs were encapsulated in 20 μL hydrogels with 500 ng of cys-IFN-γ. Following 4 days of culture, conditioned media was subjected to cytokine analysis. N=6. Error bars±SEM. One-way ANOVA, * p<0.05, ** p<0.01, *** p<0.001, **** p<0.0001.

FIG. 12 hMSC-based inhibition of monocyte differentiation is mediated primarily through IDO. After 7 days in dendritic cell differentiation conditions either with or without co-culturing with hMSCs encapsulated within cys-IFN-γ hydrogels and with or without IDO or PGE2 inhibitors 1-methyl tryptophan (1-MT) or NS-398 respectively. 10,000 cells were analyzed for each condition.

FIG. 13 Regeneration of colonic wounds in NSG mice. Quantification of colonic wound closure at day 5 post-injury. N=3-4 mice. Error bars±SEM. One-way ANOVA **p<0.01.

FIG. 14 Immunostaining for human markers within colonic wounds at 4 weeks post-implementation. Scale bar 50 μm.

### SUMMARY OF THE INVENTION

One embodiment of the invention relates to a composition comprising a scaffold, a cell, and a licensing agent, wherein the licensing agent is covalently attached to the scaffold, and the cell is non-covalently attached to the scaffold.

In one aspect, the composition further comprises at least one linker A, wherein linker A is covalently attached to the scaffold. In one aspect, linker A is a peptide linker. In one aspect, the peptide linker comprises the amino acid sequence: GRGDSPC (SEQ ID NO:6). In still another aspect, the peptide linker comprises a cell attachment amino acid sequence. In yet another aspect, the peptide linker A comprises at least one Arg-Gly-Asp (RGD) amino acid sequence. In still another aspect, a cell is non-covalently attached to the cell attachment amino acid sequence in linker A.

In one aspect, the composition further comprises at least one linker B capable of covalently joining two or more scaffolds together. In one aspect, linker B is a peptide linker. In one aspect, the peptide linker comprises the amino acid sequence: GCRDVPMSMRGGDRCG (SEQ ID NO:7). In still a further aspect, the peptide linker comprises a protease cleavage site. In yet another aspect, the peptide linker comprises at least two cysteine residues. In another aspect, the peptide linker is covalently joined to the scaffold through at least one cysteine residue in linker B.

In one aspect, the scaffold comprises at least one cysteine-reactive moiety. In one aspect, the at least one cysteine-reactive moiety is a maleimide group.

In one aspect, the licensing agent is selected from the group consisting of a protein, a cytokine, a nucleic acid, a hormone, a polysaccharide, and a lipid.

In one aspect, the licensing agent is a protein. In another aspect, the protein comprises a free cysteine residue. In yet another aspect, the protein is covalently attached to the scaffold through at least one cysteine residue in the protein. In yet another aspect, the protein is selected from the group consisting of interferon gamma, interleukin-1 alpha, interleukin-1 beta, and tumor necrosis factor. In one aspect, the protein is interferon gamma. In still another aspect, the protein is a human interferon gamma cysteine variant. In one aspect, the human interferon gamma cysteine variant is selected from the group consisting of: (a) a human interferon gamma cysteine variant wherein a cysteine residue is inserted preceding the first amino acid of the mature protein; (b) a human interferon cysteine variant wherein a cysteine residue is inserted following the last amino acid of the mature protein; and (c) a human interferon gamma cysteine variant wherein a cysteine residue is substituted for at least one amino acid in human interferon gamma (SEQ ID NO:8) selected from the group consisting of: Q1, D2, P3, N16, A17, G18, H19, S20, D21, V22, A23, D24, N25, G26, K37, E38, E39, S40, D63, Q64, S65, 166, Q67, N83, S84, N85, K86, N97, Y98, S99, V100, T101, D102, L103, P122, A123, A124, K125, T126, G127, K128, R129, K130, R131, S132, Q133, M134, L135, F136, R137, G138, R139, R140, A141, S142, and Q143. In yet another aspect, the interferon gamma cysteine variant comprises a cysteine residue substituted for leucine at position 103 of SEQ ID NO:8. In yet another aspect, the interferon gamma cysteine variant comprises a cysteine residue substituted for glutamine at position 67 of SEQ ID NO:8. In still another aspect, the interferon gamma cysteine variant further comprising a deletion of the glutamine-1 amino acid of SEQ ID NO:8.

In one aspect, the protein is selected from the group consisting of an interleukin-1 alpha cysteine variant protein, an interleukin-1 beta cysteine variant protein, and a tumor necrosis factor cysteine variant protein.

In one aspect, the scaffold is a polyethylene glycol. In one aspect, the scaffold is a multi-armed polyethylene glycol. In still another aspect, the scaffold is a 4-armed polyethylene glycol.

In one aspect, the scaffold comprises four cysteine-reactive moieties. In another aspect, the four cysteine-reactive moieties are maleimide groups.

In one aspect, the cell is a mesenchymal stem cell.

In one aspect, the cell is an induced pluripotent stem cell. In still another aspect, the pluripotent stem cell is selected from the group consisting of an embryonic stem cell and an induced pluripotent stem cell. In one aspect, the cell is a mesenchymal stem cell and wherein the licensing agent is an interferon gamma cysteine variant. In one aspect, the interferon gamma cysteine variant stimulates mesenchymal stem cells to upregulate expression of at least one immunoactive factor selected from the group consisting of: indoleamine 2,3-dixygenase (IDO), programmed death ligand-1 (PD-L1), prostaglandin E2 (PGE2), cytokines, chemokines, CCL8, CXCL9 and CXCL10.

In one aspect, the licensing agent in the composition alters the physiological properties of the cell in the composition.

In one aspect, the composition is a hydrogel.

Another embodiment of the invention relates to a method for preparing a composition comprising a scaffold, a cell, and a licensing agent, wherein the licensing agent is covalently attached to the scaffold, and the cell is non-covalently attached to the scaffold, wherein the method comprises: step (a) attaching the licensing agent to the scaffold; step (b). attaching at least one linker A to the scaffold; step (c) attaching the cells to the scaffold; and step (d) attaching at least one linker B to the scaffold.

In one aspect of this embodiment, step (c) of attaching the cells to the scaffold occurs after step (b) but before step (d).

In one aspect of this embodiment, step (d) of attaching at least one linker B to the scaffold occurs in the presence of a disulfide reducing agent. In one aspect, the disulfide reducing agent is selected from the group consisting of dithiothreitol, beta mercaptoethanol, and Tris[2-carboxyethylphosphine]hydrochloride (TCEP).

In one aspect of this embodiment, attachment of at least one linker B to the scaffold creates a hydrogel. In one aspect, the cells are encapsulated within the hydrogel.

Another embodiment of the invention relates to a method for stimulating tissue regeneration in an animal, comprising administering to at least one damaged tissue in an animal, a composition comprising a scaffold, a cell, and a licensing agent, wherein the licensing agent is covalently attached to the scaffold, and the cell is non-covalently attached to the scaffold. In one aspect, the damaged tissue is a wound. In yet another aspect, the damaged tissue is selected from the group consisting of skin, intestine, colon, heart, lung, liver, kidney, pancreas, reproductive organ, brain, nerve, nervous tissue, bone, cartilage, and ligament. In one aspect, the composition is applied locally to the damaged tissue. In yet another aspect, the composition is injected into the damaged tissue.

Another embodiment of the invention relates to a method for treating a disease in an animal, comprising administering to an animal with a disease treatable with a composition comprising a scaffold, a cell, and a licensing agent, wherein the licensing agent is covalently attached to the scaffold, and the cell is non-covalently attached to the scaffold. In one aspect, the disease is selected from the group consisting of an inflammatory disease, an autoimmune disease, and a degenerative disease. In yet another aspect, the disease is selected from the group consisting of inflammatory bowel disease, Crohn's disease, rheumatoid arthritis, graft versus host disease, autoimmune encephalomyelitis, diabetes, systemic lupus erythematosus, heart disease, kidney disease, liver disease, neurological disease, Alzheimer's Disease, Parkinson's Disease, stroke, and Multiple Sclerosis. In still another aspect, the disease is a T cell mediated disease. In one aspect, the composition inhibits T cell proliferation. In still another aspect of this embodiment, the composition inhibits T cell effector functions. In one aspect, the disease is a white blood cell related disease. In one aspect, the white blood cell related disease is selected from the group consisting of a monocyte disease, a macrophage disease, a dendritic cell disease, a microglia disease, a T cell disease, an NK cell disease, a B cell disease, a neutrophil disease, and a granulocyte disease. In yet another aspect of this embodiment, the composition inhibits differentiation of macrophages or monocytes into dendritic cells.

Another embodiment of the invention relates to a composition comprising a scaffold, a cell, and a licensing agent, wherein the licensing agent is covalently attached to the scaffold, and the cell is encapsulated within the composition. In one aspect, the composition further comprises at least one linker B capable of covalently joining two or more scaffolds together.

Another embodiment of the invention relates to a composition comprising a scaffold, a cell, a licensing agent, and at least one linker B, wherein the licensing agent is covalently attached to the scaffold, and the at least one linker B is covalently attached to the scaffold, and wherein the cell is encapsulated within the composition. In one aspect, the at least one linker B is capable of covalently joining two or more scaffolds together. In another aspect, the composition further comprises at least one linker A capable of covalently attaching to the scaffold, wherein the at least one linker A contains a sequence or region that binds or is adhesive for at least one cell. In one aspect, the at least one cell is one host cell in a patient.

Another embodiment of the invention relates to a cell-free composition comprising a scaffold, a licensing agent, and at least one linker B, wherein the licensing agent is covalently attached to the scaffold, and the at least on linker B is covalently attached to the scaffold. In one aspect, the at least one linker B is capable of covalently joining two or more scaffolds together. In another aspect, the composition further comprises at least one linker A capable of covalently attaching to the scaffold, wherein the at least one linker A contains a sequence or region that binds or is adhesive for at least one cell. In one aspect, the at least one cell is one host cell in a patient.

### DETAILED DESCRIPTION OF THE INVENTION

As disclosed herein, the inventors have engineered an injectable synthetic hydrogel with tethered recombinant interferon-gamma (IFN-γ) that activates encapsulated human mesenchymal stem cells (hMSCs) to increase their immunomodulatory functions and avoids the need for ex vivo manipulation. Tethering IFN-γ to the hydrogel increases retention of IFN-γ within the biomaterial while preserving its biological activity. hMSCs encapsulated within hydrogels with tethered IFN-γ exhibited significant differences in cytokine secretion and showed a potent ability to halt activated T-cell proliferation and monocyte-derived dendritic cell differentiation compared to hMSCs that were pre-treated with IFN-γ, and untreated hMSCs. Importantly, hMSCs encapsulated within hydrogels with tethered IFN-γ accelerated healing of colonic mucosal wounds in mice. This novel approach for licensing hMSCs with IFN-γ can enhance the clinical translation and efficacy of hMSC-based therapies.

With the impetus for cell therapies to be translated into the clinic, hMSCs have been evaluated in nearly 500 clinical trials (Squillaro T, et al. Clinical Trials With Mesenchymal Stem Cells: An Update. Cell Transplant. 2016; 25:829-48). While these cells were initially pursued for their differentiation potential, recent evidence, including their effects in treating inflammatory diseases such as GvHD and Crohn's disease, support their use for their immunomodulatory properties (Klinker M W, Wei C H. Mesenchymal stem cells in the treatment of inflammatory and autoimmune diseases in experimental animal models. World J Stem Cells. 2015; 7:556-67; Wang L T, et al. Human mesenchymal stem cells (MSCs) for treatment towards immune- and inflammation-mediated diseases: review of current clinical trials. J Biomed Sci. 2016; 23:76). Nonetheless, the success of these clinical trials in treating inflammatory diseases has been mixed with approximately half of patients treated with hMSCs showing little to no improvement (Forbes G M, et al. A phase 2 study of allogeneic mesenchymal stromal cells for luminal Crohn's disease refractory to biologic therapy. Clin Gastroenterol Hepatol. 2014; 12:64-71; Le Blanc K, et al. Mesenchymal stem cells for treatment of steroid-resistant, severe, acute graft-versus-host disease: a phase II study. Lancet. 2008; 371:1579-86; Resnick I B, et al. Treatment of severe steroid resistant acute GVHD with mesenchymal stromal cells (MSC). Am J Blood Res. 2013; 3:225-38; Panes J, et al. Expanded allogeneic adipose-derived mesenchymal stem cells (Cx601) for complex perianal fistulas in Crohn's disease: a phase 3 randomised, double-blind controlled trial. Lancet. 2016; 388:1281-90). Therefore, there is significant need for increasing the efficacy of these stem cell-based therapies and specifically, increasing the immunomodulatory properties of hMSCs. Licensing hMSCs with IFN-γ increases their immunomodulatory properties in in vitro and in vivo systems (Krampera M. Mesenchymal stromal cell ‘licensing’: a multistep process. Leukemia. 2011; 25:1408-14). However, the need for ex vivo manipulation of hMSCs with IFN-γ raises considerable barriers including increased costs, clearing regulatory hurdles, and establishing rigorous and reliable cell handling practices that impact clinical translation (Heathman T R, et al. The translation of cell-based therapies: clinical landscape and manufacturing challenges. Regen Med. 2015; 10:49-64). Engineering a biomaterial that can license hMSCs without the need for ex vivo manipulation can significantly enhance the translation of hMSC-based stem cell therapies.

Previous research has described the conjugation of bioactive proteins to biomaterials scaffolds to boost stem cell activities (Cosgrove B D, et al. N-cadherin adhesive interactions modulate matrix mechanosensing and fate commitment of mesenchymal stem cells. Nat Mater. 2016; 15:1297-306; Li H, et al. In vivo assessment of guided neural stem cell differentiation in growth factor immobilized chitosan-based hydrogel scaffolds. Biomaterials. 2014; 35:9049-57; Li R, et al. Self-assembled N-cadherin mimetic peptide hydrogels promote the chondrogenesis of mesenchymal stem cells through inhibition of canonical Wnt/beta-catenin signaling. Biomaterials. 2017; 145:33-43; Zhang K, et al. Adaptable hydrogels mediate cofactor-assisted activation of biomarker-responsive drug delivery via positive feedback for enhanced tissue regeneration. Adv Sci. 2018; 5:1800875). Disclosed herein, is a novel method for licensing hMSCs by functionalizing a PEG-based hydrogel with a biologically active form of IFN-γ. To assess the functionality and efficacy of this platform, two general concepts were tested: 1) whether the scaffold modification elicited a response in scaffold-encapsulated hMSCs, and 2) whether the effect imparted onto the hMSCs generated secondary effects on immune cells. hMSCs encapsulated within IFN-γ-presenting hydrogels exhibited similar or increased expression of both cell-licensing markers IDO and PD-L1 compared to hMSCs that were pre-licensed with soluble IFN-γ. Furthermore, hMSCs encapsulated within IFN-γ-presenting hydrogels showed a potent ability to inhibit both activated human T-cell proliferation and monocyte-derived dendritic cell differentiation. Importantly, the inhibition of dendritic cell differentiation imparted by hMSCs encapsulated within IFN-γ-tethered hydrogels was greater than that of encapsulated hMSCs that were licensed with soluble IFN-γ prior to co-culture. This increased effect for the tethered IFN-γ is likely due to the increased duration of licensing as the tethered form is present throughout the co-culture period while the unbound IFN-γ will be washed away. In addition to the increased duration, the tethered form of IFN-γ can also result in higher local concentrations of IFN-γ surrounding the encapsulated hMSCs compared to the unbound form.

Within a functional model, hMSCs encapsulated in IFN-γ presenting hydrogels exhibited significantly higher levels of mucosal wound closure compared to untreated controls as well as wounds treated with hMSCs in hydrogels. This finding supports the notion that the effects imparted by licensing hMSCs elicits a functional response in vivo.

One embodiment of the invention is a composition comprising a scaffold, a cell, and a licensing agent, wherein the licensing agent is attached to the scaffold, and the cell is attached to the scaffold. The licensing agent may be covalently or non-covalently attached to the scaffold. Preferably, the licensing agent is covalently attached to the scaffold. As used herein, attaching the licensing agent to the scaffold is also referred to as tethering the licensing agent to the scaffold. The cell may be covalently or non-covalently attached to the scaffold. Preferably, the cell is non-covalently attached to the scaffold. Preferably, the composition further comprises at least one linker A that is capable of covalently attaching to the scaffold and which contains a sequence or region that binds or is adhesive for the cell. Most preferably the cells are attached to the scaffold indirectly by binding to a linker A that is attached to the scaffold. Preferably, the composition further comprises a linker B that is capable of covalently attaching to two or more scaffolds to create a multi-scaffold structure. Optionally, linker B can comprise a sequence that allows linker B to be degraded. Preferably, degradation of linker B allows the cells in the composition to be released from the composition.

Another embodiment is a composition comprising a scaffold, a cell, and a licensing agent, wherein the licensing agent is attached to the scaffold, and the cell is incorporated into, entrapped, or encapsulated within the composition through the process of linker B covalently attaching to two or more scaffolds to create a multi-scaffold structure.

A further embodiment is a cell-free composition comprising a scaffold, a licensing agent, and at least one linker B that is capable of covalently attaching to two or more scaffolds to create a multi-scaffold structure. Optionally, linker B can comprise a sequence that allows linker B to be degraded. Preferably, degradation of linker B allows the licensing agent in the composition to be released from the composition. Optionally, the composition can comprise at least one linker A that is capable of covalently attaching to the scaffold and which contains a sequence or region that binds or is adhesive for at least one host cell to facilitate infiltration and retention of host cells within the composition. Most preferably the host cells are attached to the scaffold indirectly by binding to a linker A that is attached to the scaffold.

The scaffold of the composition can be any polymer. Preferably the polymer is a hydrophilic polymer. More preferably the scaffold is a polyethylene glycol (PEG). Even more preferably the scaffold is a PEG that contains at least two reactive groups. As used herein, the term “reactive group” has the same meaning as “reactive moiety”. Even more preferably, the scaffold is a multi-armed PEG, which is a PEG containing two or more PEG arms, each of which terminates in a reactive group. A multi-armed PEG of the invention can comprise any whole number of arms, provided that the multi-armed PEG comprises at least two arms. Thus, the multi-armed PEG of the invention can have 2 arms, 3 arms, 4 arms, 5 arms, 6 arms, 7 arms, 8 arms, 9 arms, 10 arms, 15 arms, 20, arms, 25 arms, 30 arms, 35 arms, 40 arms, 45 arms, 50 arms, 55 arms, 60 arms, 65 arms, 70 arms, 75 arms, 80 arms, 85 arms, 90 arms, 95 arms or 100 arms. In one aspect, the multi-armed PEG is between 2 and 20 arms. In still another aspect, the multi-armed PEG is between 2 and 8 arms. A preferred multi-armed PEG is a 4-armed PEG. The reactive group on the PEG can be a cysteine-reactive group, an amine reactive group, a carboxyl (COOH) reactive group, or a carbohydrate-reactive group. Preferred cysteine reactive groups include maleimide, vinyl-sulfone, thiol, and iodoacetamide groups. Preferred amine reactive groups are those capable of reacting with an amine group, such as a lysine amino acid in a peptide or protein or the amino-terminus of a peptide or protein. Examples of amine-reactive groups include but are not limited to N-hydroxysuccinimide (NHS) groups, aldehyde groups, and p-nitrophenyl carbonate groups. Examples of carboxyl-reactive groups include but are not limited to amine groups. Examples of carbohydrate-reactive groups include but are not limited to aminoxy groups and hydrazide groups.

The multi-armed PEG of the composition can range in size from 5 kilodaltons to 100 kilodaltons. Preferably the size of the PEG ranges from 5 kilodaltons to 40 kilodaltons. A preferred size of the PEG is 20 kilodaltons.

The cell that is part of the composition can be any type of cell. Preferably the cell is a mammalian cell. Preferred types of mammalian cells are mesenchymal stem cells and plutipotent stem cells. The pluripotent stem cell can be an embryonic pluripotent stem cell or an induced pluripotent stem cell. Preferred mammalian cells are human cells.

Linker A of the scaffold can be a peptide, a nucleic acid, an oligosaccharide, or a lipid. A preferred linker A is a peptide. A preferred linker A comprises at least 4 amino acids and less than 100 amino acids. A more preferred linker A comprises at least 4 amino acids and less than 20 amino acids. Preferably linker A comprises a peptide that contains one or more cysteine residues. Linker A may attach to the scaffold via the one or more cysteine residues in the linker. Preferably, linker A comprises a sequence or region that is capable of binding a cell. For this reason linker A can also be referred to as an adhesive linker. The cell can be bound non-covalently to linker A or covalently to linker A. Preferably, the cell is bound non-covalently to linker A. A preferred linker A capable of binding a cell non-covalently is a peptide comprising one or more RGD (arginine-glycine-aspartic acid) sequences, which bind to extracellular proteins, e.g., integrins, on the surfaces of cells. Other peptides that bind cells or are adhesive for cells and which can be substituted for or be used in combination with the RGD sequence include:
cyclo (N-Me-VRGDf)-cyclo(MeVal-Arg-Gly-Asp-d-Phe) (SEQ ID NO:1);
RGD-4C-Cys-Phe-Cys-Asp-Gly-Arg-Cys-Asp-Cys (SEQ ID NO:2);
c(RGDyK)-cyclo(Arg-Gly-Asp-d-Tyr-Lys) (SEQ ID NO:3);
c(RGDfC)-cyclo(Arg-Gly-Asp-d-Phe-Cys) (SEQ ID NO:4);
c(RGDfK)-cyclo(Arg-Gly-Asp-d-Phe-Lys) (SEQ ID NO:5).

An alternative preferred linker A capable of binding a cell non-covalently is a peptide comprising one or more NGR (asparagine-glycine-arginine) sequences, which also bind to extracellular integrin proteins on the surfaces of cells. A most preferred linker A is a peptide that comprises an RGD sequence and a cysteine residue. One most preferred linker A comprises the amino acid sequence GRGDSPC (SEQ ID NO:6). Another preferred linker A comprises a peptide that contains one or more lysine residues. Linker A can attach to the scaffold via the one or more lysine residues in the linker.

Linker B of the scaffold can be a peptide, a nucleic acid, an oligosaccharide, or a lipid. A preferred linker B is a peptide. A preferred linker B comprises at least 2 amino acids and less than 100 amino acids. A more preferred linker B comprises at least 5 amino acids and less than 20 amino acids. Preferably, linker B comprises a peptide that contains two or more amino acids capable of attaching to the scaffold. The two or more amino acids capable of attaching to the scaffold can be cysteine residues or lysine residues. Linker B can contain two cysteine residues, two lysine residues or a mixture of cysteine residues and lysine residues. A preferred linker B comprises a peptide containing two cysteine residues (referred to as a bicysteine peptide). Optionally, linker B can contain a sequence or region that may be cleaved enzymatically or non-enzymatically, resulting in partial or complete degradation of linker B. A linker B that can be cleaved enzymatically or non-enzymatically is also referred to as a degradable linker. Partial or complete degradation of linker B permits degradation of the multi-scaffold structure and release of the cell from the composition. A preferred degradable linker B comprises one or more amino acid sequences that are cleavable by a protease. A preferred degradable linker B comprises the amino acid sequence VPM (valine—proline—methionine), which is cleaved by matrix metalloproteinase (MMP)-1 and MMP-2 proteases, and collagenases. A most preferred degradable linker B has the amino acid sequence GCRDVPMSMRGGDRCG (SEQ ID NO:7) which is cleaved by MMP-1, MMP-2 and collagenases.

The licensing agent of the composition can comprise a protein, a cytokine, a hormone, a nucleic acid, a polysaccharide, a lipid, or any bioactive substance capable of altering one or more physiological properties of the cell within the composition. A preferred licensing agent is a protein. A more preferred licensing agent is a protein selected from the group consisting of interferon gamma, interleukin-1 alpha, interleukin-1 beta, and tumor necrosis factor. A most preferred licensing agent is interferon gamma. A preferred licensing agent comprises a reactive group capable of attaching to the scaffold. The licensing agent can be attached to the scaffold covalently or non-covalently. Preferably, the licensing agent is covalently attached to the scaffold. One preferred licensing agent is a protein containing a “free” cysteine, i.e., a cysteine not involved in a disulfide bond. If a licensing agent protein does not contain a native free cysteine residue, a free cysteine can be added to the licensing agent protein by one or more of the following methods: by insertion of a cysteine prior to the first amino acid of the mature protein, by insertion of a cysteine following the last amino acid of the protein, by insertion of a cysteine between two amino acids in the protein, or by substitution of cysteine for a non-cysteine amino acid in the protein. A free cysteine may also be introduced into the licensing agent protein by substitution a non-cysteine amino acid for a native cysteine residue that normally participates in a disulfide bond in the protein licensing agent. Preferred non-cysteine amino acids that can be substituted for a native cysteine in a disulfide bond include but are not limited to serine, alanine, and glycine. A protein in which one or more free cysteines have been added to the protein is referred to as cysteine variant protein.

Preferably, the licensing agent in the composition is capable of altering the physiological properties of the cell in the composition. Examples of physiological properties that can be altered in the cell include immunomodulatory properties, proliferative properties, antigen reactivity properties, gene expression properties, secretion properties, including secretion of cytokines and/or other bioactive substances, and differentiation properties. The altered physiological properties of the cell can be increased or decreased by the licensing agent. An example of a differentiation property that can be altered in a cell is the differentiation of a monocyte or macrophage into a dendritic cell. A preferred cell in the composition is a mesenchymal stem cell and a preferred licensing agent in the composition is an interferon gamma cysteine variant protein. The interferon gamma licensing agent can stimulate the mesenchymal stem cell to upregulate expression of immunoactive factors including but not limited to: indoleamine 2,3-dixygenase (IDO), programmed death ligand-1 (PD-L1), prostaglandin E2 (PGE2), cytokines, chemokines such as CCL8, CXCL9 and CXCL10, MCP-1, M-CSF-1, VEGF, SDF1 (CXCL12), TGF-beta, B7 family immune checkpoints, MCP-1 (CC12) among other bioactive factors.

A preferred licensing agent is interferon gamma. A most preferred licensing agent is human interferon gamma (SEQ ID NO:8). Most animal interferon gamma proteins, including human interferon gamma, do not contain any native cysteine residues. Methods for creating biologically active cysteine variant interferon gamma proteins are described in U.S. Pat. Nos. 9,296,804, 8,618,256, 8,617,531, 7,964,184 and 7,959,909 (each are herein incorporated by reference). A cysteine residue can be added to human interferon gamma by insertion preceding the first amino acid of the mature protein or by insertion following the last amino acid of the mature protein. A cysteine residue can be added to human interferon gamma by insertion between two adjacent amino acids within the primary amino acid sequence of interferon gamma as long as the biological activity or protein's conformation is not significantly affected by the cysteine insertions. A cysteine residue can be substituted for at least one amino acid in human interferon gamma having SEQ ID NO:8 selected from the group consisting of: Q1, D2, P3, N16, A17, G18, H19, S20, D21, V22, A23, D24, N25, G26, K37, E38, E39, S40, D63, Q64, S65, 166, Q67, N83, S84, N85, K86, N97, Y98, S99, V100, T101, D102, L103, P122, A123, A124, K125, T126, G127, K128, R129, K130, R131, S132, Q133, M134, L135, F136, R137, G138, R139, R140, A141, S142, and Q143. Preferred human interferon gamma cysteine variant proteins contain a cysteine substitution for the L103 amino acid (referred to as L103C) or a cysteine substitution for the Q67 amino acid (referred to as Q67C). These interferon gamma cysteine variants can be incorporated into the native interferon gamma sequence or in variants in which Q1 is deleted, or D2 is deleted, or Q1 and D2 are deleted or changed to non-glutamine or non-aspartic acids, respectively. Possible substitutions include amino acids such as alanine, arginine, aspartic acid (for Q1 only), asparagine, glutamic acid (for D2 only), glycine, histidine, isoleucine, leucine, lysine, methionine, phenylalanine, proline, serine, threonine, tryptophan, tyrosine, or valine). These interferon gamma cysteine variants also can be incorporated into the native interferon gamma sequence or in variants comprising a methionine inserted preceding the Q1 amino acid or a methionine inserted preceding the D2 amino acid if the Q1 amino acid is deleted.

In a cell-less composition, the licensing agent of the composition can comprise a protein, a cytokine, a hormone, a nucleic acid, a polysaccharide, a lipid, or any bioactive substance capable of altering one or more physiological properties of at least one host cell within a patient. The host cell within the patient may be a patient's own cell or a cell transplanted into the patient from another subject.

Another embodiment of the invention comprises a method for preparing the composition disclosed herein. The method comprises: step (a) attaching the licensing agent to the scaffold, step (b) attaching linker A to the scaffold; step (c) attaching the cells to the scaffold through binding of the cells to linker A and/or entrapping the cells in the hydrogel network; and step (d) attaching linker B to the scaffold.

Preferably, the cells are incorporated into the composition by adding the cells prior to step (d). Preferably, the cells are added after step (b) but before step (d). Optionally, step (d) can occur in the presence of a disulfide reducing agent. The disulfide reducing agent can be any compound capable of reducing a disulfide bond. Examples of preferred disulfide reducing agents include dithiothreitol, beta mercaptoethanol, and Tris[2-carboxyethylphosphine]hydrochloride (TCEP). Preferably step (d) occurs after steps (a), (b), and (c).

Another embodiment of the invention comprises a method for preparing the cell-less composition disclosed herein. The method for preparing the cell-less composition comprises the same steps as described above for preparing the composition except that steps (b) and (c) are omitted; i.e., the method for preparing the cell-less composition comprises: Step A attaching the licensing agent to the scaffold, and step (d) attaching linker B to the scaffold. Preferably step (d) occurs after step (a). Optionally, step (d) can occur in the presence of a disulfide reducing agent. The disulfide reducing agent can be any compound capable of reducing a disulfide bond. Examples of preferred disulfide reducing agents include dithiothreitol, beta mercaptoethanol, and Tris[2-carboxyethylphosphine]hydrochloride (TCEP). Optionally, in cases where a linker A is desired within the cell-less composition, the method for preparing the cell-less composition comprises: step (a) attaching the licensing agent to the scaffold, step (b) of attaching at last one linker A to the scaffold, and step (d) attaching at least one linker B to the scaffold. Preferably, at least one linker A is attached to the scaffold after step (a) and before step (d).

Optionally, the composition can be allowed to gel after addition of at least one linker B to create a hydrogel. Gelling may occur at any temperature and for any length of time. Preferred gelling temperatures range from 0° C. to 50° C., more preferably from 15° C. to 50° C., and even more preferably from 20° C. to 37° C. A preferred gelling temperature is 37° C. Preferred gelling times range from 10 seconds to 120 minutes, more preferably from 1 minute to 30 minutes, and even more preferably from 5 minutes to 15 minutes. A preferred gelling time is 10 minutes. After gelling, phosphate buffered saline or cell culture media may be added to the composition. The composition can be used immediately or stored for later use. The composition can be stored at temperatures ranging from less than −70° C. to 50° C., more preferably at temperatures ranging from less than −70° C. to 20° C., and even more preferably from less than −70° C. to 8° C. When stored at or below 0° C., a cryoprotectant can be added to the composition to help maintain cell viability. Examples of useful cryoprotectants include but are not limited to dimethyl sulfoxide and glycerol. A preferred final concentration of a cryoprotectant in the composition ranges from 1% to 20% (volume:volume if a liquid), and more preferably from 5% to 15% (volume:volume if a liquid). A most preferred croprotectant concentration is 10% (volume/volume if a liquid).

The composition can contain varying amounts of the scaffold, linker A, linker B, the licensing agent, and cells, if desired. The concentration of linker A in the composition can range from 0.01 mM to 100 mM. Preferably, the concentration of linker A ranges from 0.1 mM to 10 mM. A preferred final concentration of linker A in the composition is 1.0 mM. The concentration of licensing agent in the composition can range from 0.1 μg/mL to 100 mg/mL, more preferably, from 1 μg/mL to 10 mg/mL, and even more preferably from 10 μg/mL to 1 mg/mL. A preferred concentration of the licensing agent is 25 μg/mL. Preferably the amount of licensing agent added to the scaffold should be less than the amount that would attach to all of the reactive groups of the scaffold. Preferably the amount of linker A added to the scaffold should be less than the amount that would attach to all of the reactive groups of the scaffold. Most preferably, the amount of the licensing agent and linker A added to the scaffold should be less than the amount that would attach to all of the reactive groups of the scaffold. A preferred concentration of linker B used for preparing the composition can be calculated by matching the number of reactive groups (free cysteines in the linker B solution, or lysines in the linker B solution) to the number of residual reactive groups (e.g., maleimides) in the scaffold following addition of the licensing agent and linker A to the scaffold.

A preferred composition comprises a 4-armed maleimide PEG scaffold synthesized to comprise a final concentration of 1.0 mM linker A peptide and a concentration of 25 μg/mL of an interferon gamma cysteine variant. A preferred concentration of linker B used for the synthesis of the composition can be calculated by matching the number of reactive groups (cysteine and lysines in the linker B solution) to the number of residual reactive groups (e.g., maleimides) in the scaffold following addition of the licensing agent and linker B to the scaffold.

Preferred weight concentrations (weight/volume) of the scaffold polymer in the composition ranges from 0.1 to 20 percent. More preferred weight concentrations of the scaffold polymer in the hydrogel composition range from 3 to 15 percent. Even more preferred weight concentrations of the scaffold polymer in the hydrogel composition ranges from 4 to 6 percent.

Preferred number of cells in the composition ranges from 0.1 to 50 million per mL. More preferred numbers of cells in the composition range from 1 million per mL to 5 million per mL. A further embodiment of the invention is a method for using the composition to stimulate tissue regeneration in an animal, the method comprising administering the composition to one or more damaged tissues in an animal. The damaged tissue can be any tissue or organ, including but not limited to skin, intestine, colon, heart, lung, liver, kidney, pancreas, reproductive organ, brain, nerve, nervous tissue, bone, cartilage, and ligament. The tissue damage can result from any number of physiological insults, including but not limited to a wound, tissue necrosis, dead or dying cells, or inability of cells within the tissue to function normally. The composition can be applied to the damaged organ by any means known to an expert in the art. Preferred methods for applying the composition to the damaged tissue include but are not limited to surface application, injection, local application, topical application, subcutaneous application, and implantation of precast scaffold.

A preferred method is local application of the composition to the damaged tissue or damaged organ. The composition can be applied topically or locally to an exposed surface such as a wound on skin to promote faster or more complete healing of the wound. The composition can be applied to the damaged tissue or damaged organ using an endoscope, colonoscope, or other instrument to promote regeneration of the damaged tissue or damaged organ, or faster or more complete healing of the damaged tissue or damaged organ. Alternatively, the composition can be injected directly into a damaged tissue. For example, the composition can be injected into an intestinal fissure in a subject with one or more intestinal fissures to promote faster or more complete healing of the fissures.

A further embodiment of the invention is a method for using the composition to treat a disease in an animal, the method comprising administering the composition to an animal with a disease treatable with the composition. Examples of the types of diseases for which the composition can be useful for treating include but are not limited to an inflammatory disease, an autoimmune disease, and a degenerative disease. Examples of specific diseases for which the composition can be useful to treat include, but are not limited to inflammatory bowel disease, Crohn's disease, rheumatoid arthritis, graft versus host disease, autoimmune encephalomyelitis, diabetes, systemic lupus erythematosus, heart disease, kidney disease, liver disease, neurological disease, Alzheimer's Disease, Parkinson's Disease, stroke, and Multiple Sclerosis.

A further embodiment of the invention is a method for treating a T cell mediated disease in an animal, the method comprising administering the composition to an animal with a T cell mediated disease. The T cell mediated disease can be an autoimmune disease, an inflammatory disease, or a cancer disease. The composition can inhibit or stimulate T cell proliferation, or stimulate or inhibit T cell effector functions. Examples of T cell effector functions that can be stimulated or inhibited by the composition include but are not limited to macrophage activation, B cell activation, B cell differentiation, stimulation of B cell antibody secretion, target cell killing, secretion of cytotoxic effector molecules such as perforins, granzymes, and fas ligand, and secretion of cytokines such as interferon gamma, tumor necrosis factor alpha, tumor necrosis factor beta, GM-CSF, CD40 ligand, fas ligand, IL-2, IL-3, IL-4, IL-5, IL-10, eotaxin, and transforming growth factor beta. Target cells killed by T cells or macrophages activated by T cells include but are not limited to cancer cells, virus infected cells, bacteria infected cells, fungi infected cells and other microbe infected cells. Inappropriate activation of T cells may result in killing or damage to healthy cells, resulting in an autoimmune disease.

A further embodiment of the invention is a method for treating a white blood cell related disease in an animal, the method comprising administering the composition to an animal with a white blood cell related disease. White blood cells include but are not limited to monocytes, macrophages, dendritic cells, microglia, T cells, B cells, and natural killer cells. The white blood cell related disease can be an autoimmune disease, an inflammatory disease, a cancer disease, or an immune suppressive disease. The composition can inhibit or stimulate white blood cell proliferation or inhibit or stimulate white blood cell effector functions. Examples of white blood cell effector functions that can be inhibited or stimulated by the composition include but are not limited to the T cell effector functions described above, as well as macrophage activation, killing of cancer cells, killing of virus infected cells, killing of bacteria infected cells, killing of fungi infected cells, killing of other microbe infected cells, secretion of cytokines or other bioactive molecules, antigen presentation, activation of T cells, activation of B cells, stimulation of T cell immune responses, stimulation of B cell immune responses, and stimulation of tissue repair.

As demonstrated in the Examples below, a fully synthetic and injectable scaffold was engineered to present a covalently-bound form of IFN-γ for providing a persistent licensing cue for activation of hMSCs. Further, hMSCs encapsulated within this scaffold are shown to elicit enhanced immunomodulatory properties and repair of colonic wounds in both immunocompromised and immunocompetent mouse models. The results establish a simple, translatable, biomaterials-based strategy to enhance the immunomodulatory activities of hMSCs.

The following experimental results are provided for purposes of illustration and are not intended to limit the scope of the invention.

### EXAMPLES

### Example 1

### Materials and Methods for Examples 2-5

### Cell Culture

All human cell isolation and culture procedures were performed following IRB-approved protocols. Human mesenchymal stem cells were acquired from the NIH Resource Center at Texas A&M University and confirmed as hMSCs (Dominici M, et al. Minimal criteria for defining multipotent mesenchymal stromal cells. The International Society for Cellular Therapy position statement. Cytotherapy. 2006; 8:315-7. Briefly, cells were obtained from healthy donors via bone marrow aspirate, followed by density centrifugation for mononuclear cells and selected for adherent culture. Cells were screened for colony forming units, cell growth, and differentiation into fat and bone using standard assays. Flow cytometry analyses confirmed that cells were positive for CD90, CD105, CD73a and negative for CD34, CD11b, CD45, CD19. Received frozen stocks were thawed and grown in α-MEM containing 16% fetal bovine serum (FBS), 2 mM L-glutamine and 100 U/mL penicillin/streptomycin (ThermoFisher, MA). Human CD4+ T-cells were purified from frozen leukapheresis samples from Emory University through negative selection with a CD4 T-cell isolation kit according to the manufacturer's instructions (Biolegend, CA). Human monocytes were purified from peripheral blood mononuclear cells (PBMCs). Briefly, peripheral blood was diluted 1:1 with PBS containing 2% FBS after which the peripheral blood mononuclear cells (PBMCs) were separated via density gradient centrifugation (specific gravity: 1.077 g/mL, Stemcell Technologies, Canada). The isolated PBMCs were washed and subjected to monocyte purification using the EasySep human monocyte isolation kit (Stemcell Technologies, Canada) according to the manufacturer's instructions. All cell culture was conducted at 37° C. in a 5% CO₂ atmosphere.

### PEG Hydrogel Synthesis and IFN-γ Functionalization

Recombinant IFN-γ engineered to express a surface-exposed cysteine at amino acid position 103 (cys-IFN-γ) was expressed in E. coli and purified by ion exchange chromatography using a S-Sepharose column as previously described (Fam C M, et al. PEGylation improves the pharmacokinetic properties and ability of interferon gamma to inhibit growth of a human tumor xenograft in athymic mice. J Interferon Cytokine Res. 2014; 34:759-68). Four-arm maleimide-end functionalized PEG macromer (PEG-4MAL 20 kDa MW, Laysan Bio, AL, >95% purity, >95% end-functionalization) was functionalized with cys-IFN-γ for 1 hr at room temperature in phosphate buffered saline at pH=7.4. The macromer was further functionalized with RGD peptide (GRGDSPC (SEQ ID NO:6), final concentration 1.0 mM) (GENSCRIPT®, NJ). The functionalized macromers were crosslinked using a mixture of the bi-cysteine peptide VPM (GCRDVPMSMRGGDRCG (SEQ ID NO:7)) (GENSCRIPT®, NJ) and dithiothreitol (SIGMA-ALDRICH®, MO). The concentration of cross-linker used for the synthesis of each hydrogel was calculated by matching the number of cysteines in the crosslinking solution to the number of residual maleimides following complete macromer functionalization. In certain experiments, cys-IFN-γ was substituted with the non-cysteine-containing, wild-type human recombinant IFN-γ (BIOLEGEND®, CA). Cys-IFN-γ functionalized into the PEG-4MAL hydrogel is termed cys-IFN-γ hydrogels' whereas non-cysteine-expressing IFN-γ mixed into the PEG-4MAL hydrogel precursor is termed ‘IFN-γ hydrogels’. In experiments where cells were encapsulated in hydrogels, a pre-determined number of cells were mixed with the functionalized macromer followed by crosslinking. Hydrogels were allowed to gel at 37° C. for 10 minutes before swelling in either PBS or complete cell culture media if cells were encapsulated in the hydrogel. Tethering of cys-IFN-γ onto PEG-4MAL was determined through protein gel electrophoresis on an SDS-PAGE gel followed by protein visualization with Sypro Ruby according to manufacturer's instructions (THERMOFISHER®, MA). For Western blotting, cys-IFN-γ or native IFN-γ was reacted with PEG-4MAL at room temperature for 30 min. Samples were mixed in SDS-PAGE reducing sample loading buffer and denatured at 100° C. for 5 min. 100 ng of Cys-IFN-γ, Cys-IFN-γ+PEG-4MAL, native IFN-γ, and native IFN-γ+PEG-4MAL were loaded per lane of Bolt™ 4-12% Bis-Tris Plus Gels, separated by electrophoresis, and transferred onto a nitrocellulose membrane. Blotted membrane was blocked at room temperature for 1 hr using Odyssey Blocking Buffer in TBS (LI-COR®). Primary anti-IFN-γ (1:1,000 in blocking buffer, ab25101, Abcam) was incubated on an orbital shaker at 4° C. overnight. Secondary anti-rabbit (1:10,000 in blocking buffer, IRDye 680RD goat anti-rabbit IgG, LI-COR®) was incubated on an orbital shaker at room temperature for 1 hr. Fluorescent bands were detected using the Odyssey CLx imaging system (LI-COR®).

### IFN-γ Release Kinetics

To assess IFN-γ release kinetics from hydrogels, hydrogels were synthesized with either cys-IFN-γ or IFN-γ. Hydrogels were incubated in PBS for 4 days with supernatant collected at specified time points, snap-frozen and stored at −80° C. At day 4, the PBS from all wells was removed and replaced with fresh PBS with a subset of wells having hydrogels having cys-IFN-γ, receiving PBS with 50 μg/mL collagenase (Worthington Biochemical, NJ). Supernatants were collected at specified time points for an additional 3 days, snap-frozen and stored at −80° C. At the end of the experiment, samples were thawed and the concentration of IFN-γ assessed via ELISA (BIOLEGEND®, CA).

### Bioactivity of Cys-IFN-γ

hMSCs were plated onto 24-well tissue culture plastic plates at a density of 10,000 cells/cm′. Four hr after seeding, various forms of IFN-γ were added to the cultures at a concentration of 50 ng/mL (Klinker M W, et al. Morphological features of IFN-gamma-stimulated mesenchymal stromal cells predict overall immunosuppressive capacity. Proc Natl Acad Sci USA. 2017; 114:E2598-e607). After 4 days in culture, the conditioned media was collected and frozen at −80° C. hMSCs were trypsinized, fixed, permeabilized and subjected to flow cytometric analysis on a BD Accuri C6 flow cytometer for expression of IDO and PD-L1. Conditioned media was analyzed for secreted proteins using a custom Luminex kit (R&D Systems, MN).

Cys-IFN-γ in Hydrogel-Encapsulated hMSC Culture

hMSCs were encapsulated in hydrogels containing cys-IFN-γ, IFN-γ or no IFN-γ as described above at a concentration of 5×10⁶ cells/mL. After 4 days in culture, conditioned media was collected and frozen at −80° C. Hydrogels were then degraded by incubation in 1 mg/mL collagenase in PBS for 30 min at 37° C. Cells were collected and subjected to flow cytometric analysis for expression of IDO and PD-L1. Conditioned media was analyzed for various proteins using a custom Luminex kit (R&D Systems, MN).

### IDO Activity Assay

Tryptophan is converted to kynurenine through IDO activity (Grohmann U, et al. Tolerance, DCs and tryptophan: much ado about IDO. Trends Immunol. 2003; 24:242-8). Kynurenine was quantified using a protocol previously described (Zhang Q, et al. Mesenchymal stem cells derived from human gingiva are capable of immunomodulatory functions and ameliorate inflammation-related tissue destruction in experimental colitis. J Immunol. 2009; 183:7787-98). Briefly, 150 μL of conditioned media after 4 days of culture in specified conditions was collected and mixed with 50 μL of 30% trichloroacetic acid. This solution was then heated to 50° C. for 10 min. Solutions were then vortexed and centrifuged at 10,000 g for 5 min. 75 μL of supernatant samples were mixed with 75 μL of Ehrlich's reagent and incubated for 10 min. Absorbance was then read at 492 nm.

### T-Cell Proliferation Assay

hMSCs (1×10⁶ cells/mL) were encapsulated in hydrogels (20 μL) with cys-IFN-γ, IFN-γ, or no IFN-γ. For the pre-licensed group, hMSCs on tissue culture plastic were stimulated with IFN-γ for 48 hr prior to encapsulation in no IFN-γ hydrogels. To simulate in vivo applications in which a sink environment is present, cys-IFN-γ and IFN-γ hydrogels were washed two times over the course the first 24 hr following hydrogel synthesis. Following 48 hr of hMSC-hydrogel culture, CD4+ T-cells purified from PBMCs were resuspended in RPMI 1640 supplemented with 10% FBS, 2 mM L-glutamine, 10 mM cell-culture grade HEPES and 100 U/mL penicillin/streptomycin. CD4+ T-cells (100,000) were added to each well in a 96 well plate and stimulated with 2 μL of Dynabeads (THERMOFISHER®, MA). hMSC-encapsulated hydrogels were then transferred to wells containing the CD4+ T-cells and co-cultured for an additional 4 days. Eight hr prior to the end of culture, EdU was added to the media. At the end of 4 days, hydrogels were removed from the co-culture, T-cells were collected, fixed and permeabilized. T-cells were stained for DAPI and EdU that was incorporated into the T-cells upon proliferation was stained by using a Click-iT EdU kit (THERMOFISHER®, MA) according to manufacturer's instructions. Stained T-cells were imaged using a Nikon C2 confocal microscope and the proliferation of T-cells as quantified by taking the ratio of EdU+/total cells was performed using a custom ImageJ macro. In certain experiments, 1-methyl-L-tryptophan (1-MT) (SIGMA-ALDRICH®, MO) was used to inhibit IDO activity. In these experiments, 1-MT was added to the media at the start of co-culture at a concentration of 1.0 mM 1-MT. T-cells were subjected to the same EdU staining protocol as described above.

### Monocyte-Derived Dendritic Cell Differentiation Assay

hMSCs were encapsulated in cys-IFN-γ, IFN-γ or no IFN-γ hydrogels (20 μL) at a concentration of 2.5×10⁶ cells/mL. For the pre-licensed group, hMSCs on TCP were stimulated with IFN-γ for 48 hr prior to encapsulation in no IFN-γ hydrogels. To simulate in vivo applications in which a sink environment is present, cys-IFN-γ and IFN-γ hydrogels were washed two times over the course the first 24 hr following hydrogel synthesis. Hydrogels were cultured in this manner for 48 hr. Following 48 hr of hMSC-encapsulated hydrogel culture, purified human monocytes isolated from peripheral blood and monocytes (500,000) were added into wells of a 24-well plate. Monocytes were cultured in RPMI 1640 supplemented with 10% FBS, 2 mM L-glutamine, 100 U/mL penicillin/streptomycin, 50 ng/mL GM-CSF (BIOLEGEND®, CA) and 20 ng/mL IL-4 (BIOLGEND®, CA). hMSC-encapsulated hydrogels were then transferred to wells containing monocytes and co-cultured for 5 days with media changes every 2-3 days. At day 5, 100 ng/mL lipopolysaccharide (LPS) (SIGMA-ALDRICH®, MO) was added to each well to induce maturation of dendritic cells. Cells were cultured for an additional 48 hr after which the monocytes were gathered and subjected to flow cytometric analysis for CD1a, CD14, CD80 and CD86 on a BD Accuri C6 flow cytometer. In certain experiments, 1-methyl-L-tryptophan (1-MT) (SIGMA-ALDRICH®, MO) was utilized to inhibit IDO activity. In these experiments, 1-MT was added to the media at the start of co-culture at a concentration of 1.0 mM 1-MT. The differentiated monocytes were subjected to the same flow cytometric analysis as described above.

### Colonic Wound Surgery and Injections

All animal experiments were performed with the approval of the University of Michigan Animal Care and Use Committee within the guidelines of the Guide for the Care and Use of Laboratory Animals and in accordance with the US Department of Agriculture (USDA) Animal and Plant Health Inspection Service (APHIS) regulations and the National Institutes of Health (NIH) Office of Laboratory Animal Welfare (OLAW) regulations governing the use of vertebrate animals. Colonic wounds were induced in a method similar to previously published protocols (Cruz-Acuna R, et al. Synthetic hydrogels for human intestinal organoid generation and colonic wound repair. Nat Cell Biol. 2017; 19:1326-35). Briefly, male (8 weeks old) NOD-SCID IL2Rg-null (NSG) or C57/B6 mice (Jackson Laboratory) were anaesthetized by intraperitoneal injection of a ketamine (100 mg/kg)/xylazine (10 mg/kg) solution. A high-resolution miniaturized colonoscope system equipped with biopsy forceps (Coloview Veterinary Endoscope) was used to biopsy-injure the colonic mucosa at 5 sites along the dorsal artery. Wound size averaged approximately 1 mm². 50 μL hydrogel injections were performed 1 day following wounding with the aid of a custom-made device comprising a 29-gauge needle connected to a small tube. Endoscopic procedures were viewed with high-resolution (1,024×768 pixels) live video on a flat-panel color monitor. Each wound region was digitally photographed at day 1 and day 5 and resulting wound images for which the wound area was calculated by a blinded observer using ImageJ. Results for one mouse were averaged through quantification of the five colonic wounds/injections per mouse. To identify transplanted hMSC, tissue sections were immunostained with an antibody specific to human nuclear antigen (MAB1281, EMD Millipore).

### Statistics

All experiments were performed on biological replicates. Sample size for each experimental group is reported in the appropriate figure legend. Unless otherwise noted, error bars on graphs represent SEM. Comparisons among multiple groups was performed by one-way analysis of variance (ANOVA) with post-hoc Tukey tests if data did not have significant differences in standard deviation. Data with significant differences in standard deviation were subject to log transformation after which post-hoc Tukey test performed. All statistics were performed in GraphPad Prism. A p-value of <0.05 was considered significant.

### Example 2

Synthetic Hydrogels with Controlled Presentation of Tethered IFN-γ

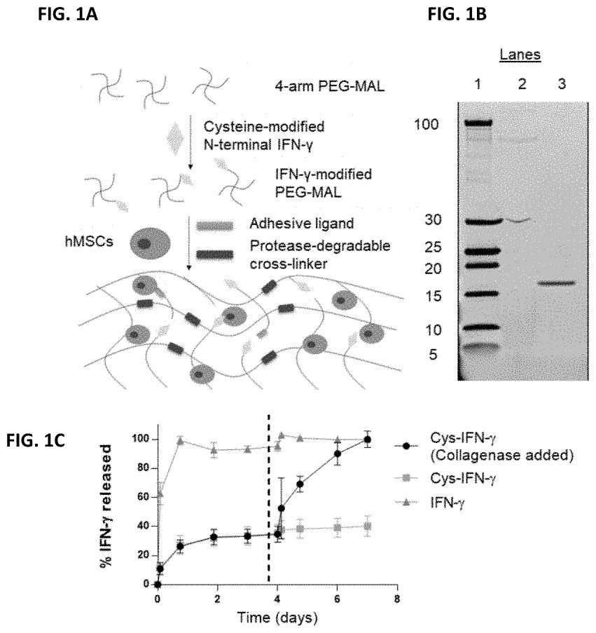

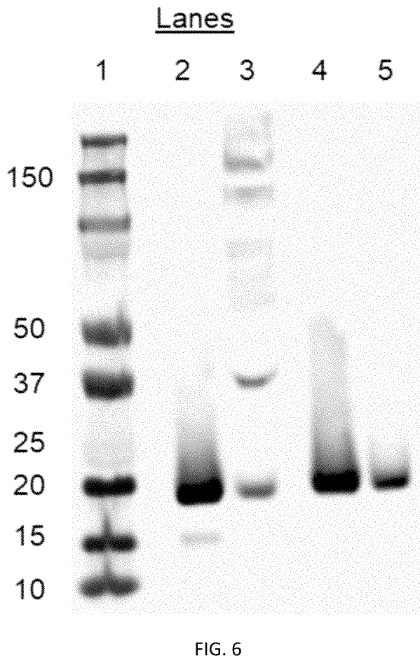

Hydrogels were engineered based on a maleimide-functionalized 4-armed poly(ethylene glycol)-based PEG macromer (PEG-4MAL) which allows for facile covalent tethering of peptides with a surface-accessible cysteine (FIG. 1A). In this system, IFN-γ is covalently tethered onto the macromer which is then incorporated into the hydrogel network. An adhesive peptide (RGD) was incorporated in the hydrogel to support cell activities and tissue integration. Cell-laden hydrogels were synthesized by mixing RGD peptide and hMSCs with PEG-4MAL followed by further reaction with a protease-degradable bicysteine peptide, which results in an insoluble and crosslinked PEG-based hydrogel sensitive to proteolytic degradation. Native human IFN-γ has no cysteines and thus no ability to conjugate onto the PEG-4MAL macromer without the addition of other linking reagents. To circumvent this, an IFN-γ variant that is genetically engineered to express a surface-available cysteine residue at amino acid position 103 (Fam C M, et al. PEGylation improves the pharmacokinetic properties and ability of interferon gamma to inhibit growth of a human tumor xenograft in athymic mice. J Interferon Cytokine Res. 2014; 34:759-68) was utilized. To verify that this IFN-γ cysteine variant could be functionalized onto the PEG-4MAL macromer, protein gel electrophoresis was performed (FIG. 1B). Cysteine-presenting IFN-γ (cys-IFN-γ) that was not reacted with PEG-4MAL and instead mixed with PBS exhibited a distinct single band at approximately 17 kDa as expected (lane 3, ladder on lane 1). When cys-IFN-γ was reacted with PEG-4MAL macromer (20 kDa), a new band appears around 30 kDa, indicating successful conjugation (lane 2). Western blot analysis was also performed to further verify the tethered nature of the cys-IFN-γ and found a similar band around 37 kDa for the cys-IFN-γ reacted with PEG-4MAL compared to the expected single band at 17 kDa for the cys-IFN-γ reacted with PBS (FIG. 6). As expected, native IFN-γ reacted with PEG-4MAL did not show a shift in molecular weight indicative of PEGylation (FIG. 6). To further confirm the tethered nature of cys-IFN-γ on the PEG-4MAL macromer, a release assay was performed in which either cys-IFN-γ or IFN-γ was reacted with PEG-4MAL macromer and crosslinked into hydrogels using the protease-degradable peptide. The hydrogels were then placed in buffer and examined for release of IFN-γ into the medium by ELISA (FIG. 1C). Hydrogels containing native IFN-γ exhibited >60% IFN-γ burst release after only 2 hr followed by complete release by 18 hr. In contrast, hydrogels containing cys-IFN-γ released ˜20% of the total incorporated IFN-γ after 2 hr and after 4 days still retained approximately 65% of total incorporated protein. The initial release of cys-IFN-γ is attributed to protein that was not tethered to the hydrogel backbone. This is not unexpected as it has been previously shown that a fraction (˜20-30%) of other proteins (e.g., VEGF) encapsulated within PEG-4MAL gels is not covalently tethered to the hydrogel and passively released in PBS (Garcia J R, et al. Integrin-specific hydrogels functionalized with VEGF for vascularization and bone regeneration of critical-size bone defects. J Biomed Mater Res A. 2016; 104:889-900). To show that the protein retained in hydrogel is related to cys-IFN-γ tethering onto the hydrogel backbone, a subset of cys-IFN-γ-containing hydrogels were incubated for 4 days in 50 μg/mL collagenase in PBS. Addition of collagenase caused degradation of the hydrogel over the course of the following three days and resulted in complete cys-IFN-γ release. Together, the protein electrophoresis and release results confirm that the cys-IFN-γ is chemically conjugated to the PEG-4MAL macromer and subsequently tethered into the crosslinked hydrogel.

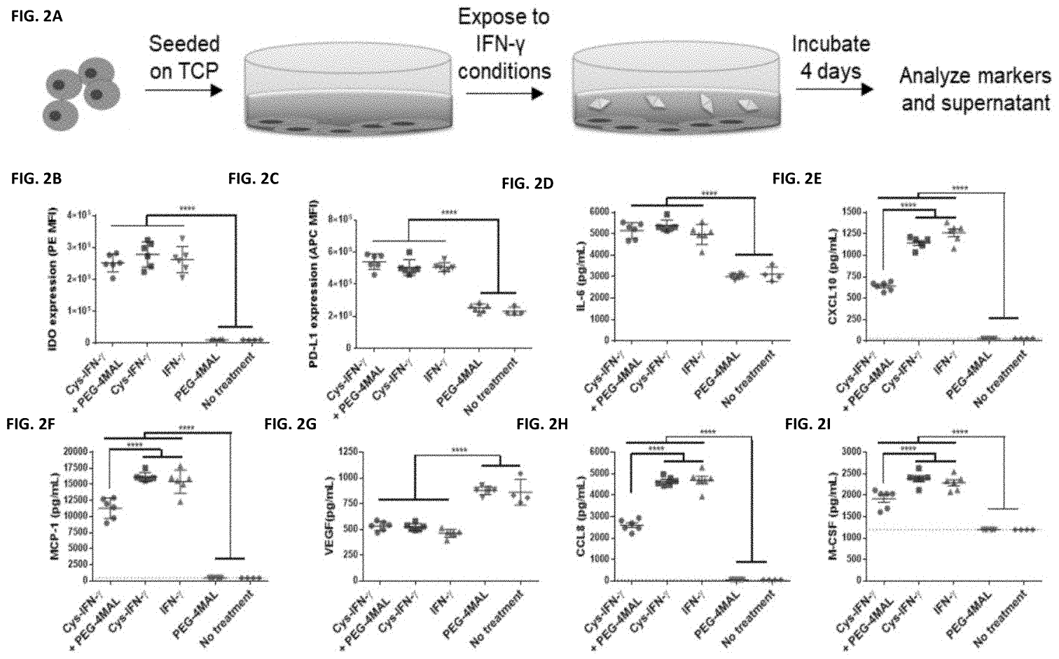

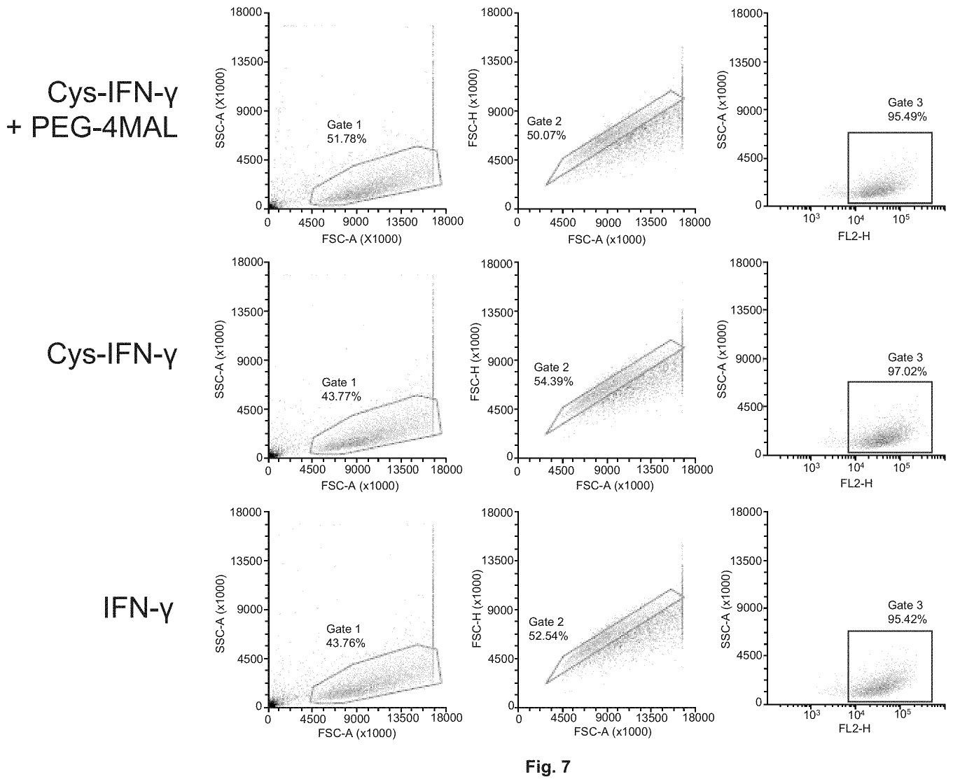

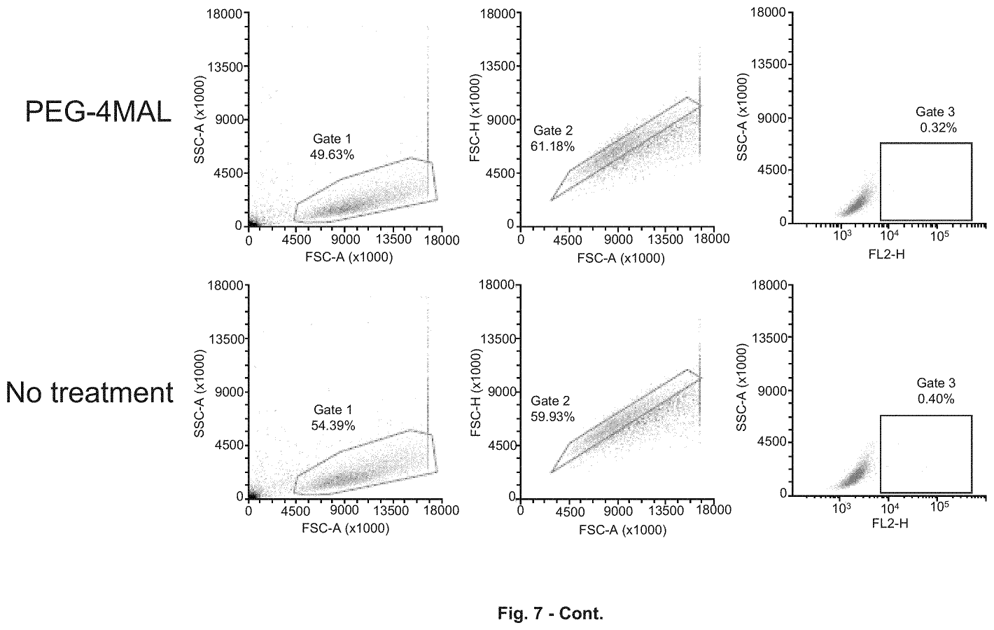

To assess whether its biological activity is affected by the chemical conjugation of the cys-IFN-γ onto PEG-4MAL macromer, hMSCs were plated on tissue-culture plastic wells and incubated in cell culture media supplemented with either cys-IFN-γ reacted with PEG-4MAL (cys-IFN-γ+PEG-4MAL), cys-IFN-γ, native IFN-γ, PEG-4MAL without IFN-γ or no treatment control for 4 days (FIG. 7, FIG. 2A). Flow cytometric analysis was then performed for IDO and PD-L1 expression and cytokine secretion assessed using a Luminex kit. hMSCs incubated with cys-IFN-γ+PEG-4MAL, cys-IFN-γ, or native IFN-γ showed significantly increased levels of IDO and PD-L1 expression as assessed by median fluorescence intensity (MFI) compared to hMSCs incubated with PEG-4MAL or cell culture media alone (FIG. 2B, 2C). Importantly, there were no differences in IDO or PD-L1 expression among hMSCs exposed to cys-IFN-γ+PEG-4MAL, cys-IFN-γ or native IFN-γ, demonstrating that cys-IFN-γ has equivalent biological activity to the native protein and that conjugation to PEG-4MAL macromer does not affect its activity. Moreover, the concentrations of secreted IL-6, CXCL10, CCL2, CCL8, and M-CSF were all significantly increased while VEGF was significantly decreased in hMSCs exposed to cys-IFN-γ+PEG-4MAL, the cys-IFN-γ or native IFN-γ compared to groups not treated with IFN-γ (FIG. 2D-I). No significant differences were noted among cys-IFN-γ+PEG-4MAL, cys-IFN-γ and native IFN-γ for IL-6. However, cys-IFN-γ+PEG-4MAL did show decreases in CXCL10, CCL2, CCL8 and M-CSF concentrations compared to cys-IFN-γ without PEG-4MAL and native IFN-γ, reflecting a slight loss in activity resulting from PEGylation. Nevertheless, the cys-IFN-γ+PEG-4MAL exhibits significantly higher activity than the negative controls.

### Example 3

Enhanced hMSC Immunoactivation in Hydrogels with Tethered IFN-γ

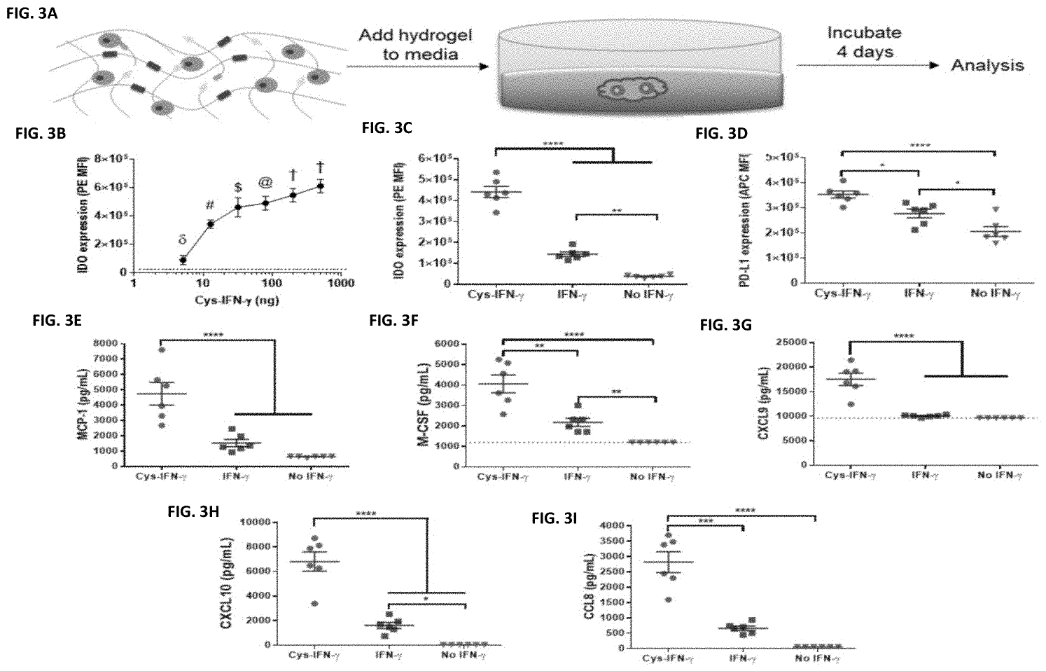

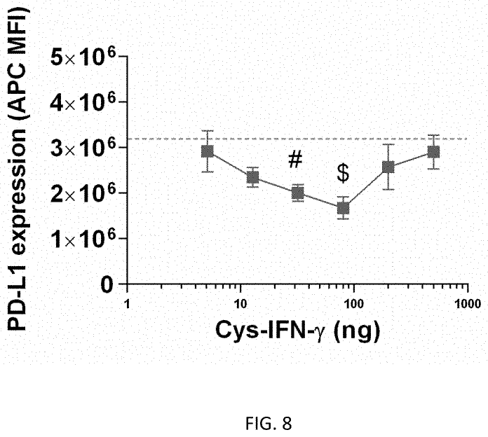

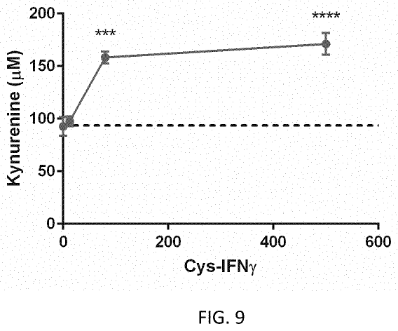

Whether hydrogels presenting cys-IFN-γ modulate the immunomodulatory phenotype of encapsulated-hMSCs was then examined (FIG. 3A). hMSCs were encapsulated in hydrogels engineered with different doses of cys-IFN-γ ranging from 0-500 ng in a 20 μL hydrogel (final cys-IFN-γ concentration 0-25 μg/mL) to assess the dose response of hMSCs to cys-IFN-γ. No differences in cell viability or growth were observed after encapsulation among hydrogel groups. Following 4 days in culture, hMSCs were subjected to flow cytometric analysis for PD-L1 (FIG. 8) and IDO (FIG. 3B). Expression of PD-L1 decreased as the concentration of cys-IFN-γ increased from 0 to 80 ng but then increased from 80 to 500 ng. While PD-L1 expression increased at doses of 80 ng of cys-IFN-γ and higher, PD-L1 expression was not significantly different at 500 ng, the highest dose tested, compared to basal expression levels. Notably, IDO expression increased with cys-IFN-γ concentration in a dose-dependent fashion with doses greater than 10 ng showing a significant increase in IDO compared to basal IDO levels (FIG. 3B). That increased IDO expression correlated with increased IDO activity was also confirmed by measuring the concentration of kynurenine, the product of tryptophan after its catalysis by IDO (FIG. 9). For subsequent studies, a concentration of 25 μg/mL of cys-IFN-γ within the hydrogel was used because this dose yielded the highest IDO expression in encapsulated hMSC.

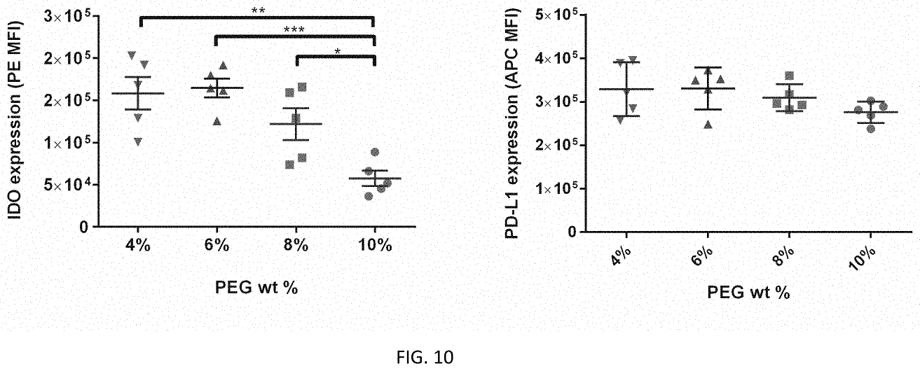

How the polymer density of the hydrogel, which controls the mechanical properties and diffusivity of the gel, influences the expression of IDO and PD-L1 for encapsulated hMSCs was then studied as polymer density may affect the availability of biological agents to encapsulated cells [Stevens M M, George J H. Exploring and engineering the cell surface interface. Science. 2005; 310:1135-8]. hMSC-laden hydrogels of differing polymer densities ranging from 4% to 10% were synthesized with a constant 25 μg/mL concentration of cys-IFN-γ. Following 4 days in culture, hMSCs were subjected to flow cytometric analysis for expression of IDO and PD-L1 (FIG. 10). Whereas no differences were noted for PD-L1 expression as a function of polymer density, hMSCs within 10% hydrogels exhibited significantly lower levels of expression of IDO compared to those in 4%, 6% and 8% hydrogels. Together, these results show that the expression of IDO is significantly influenced by the dose of cys-IFN-γ and the polymer density of the surrounding biomaterial environment. Based on these results, 6% hydrogels with 25 μg/mL IFN-γ were chosen for subsequent in vitro experiments as these conditions correlated with the highest level of hMSC-based IDO expression.

### Example 4

Cys-IFN-γ hydrogels enhance hMSC Immunomodulatory Activities

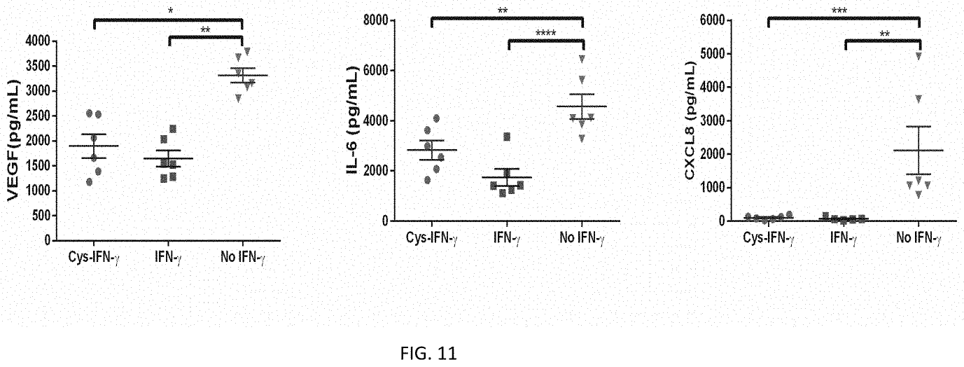

A potential advantage of presenting IFN-γ tethered to the hydrogel microenvironment is enhanced and sustained licensing compared to stimulation with soluble IFN-γ. Whether IFN-γ tethering to the hydrogel increases licensing duration compared to soluble IFN-γ was therefore studied. hMSCs were encapsulated in hydrogels with either cys-IFN-γ, IFN-γ or no IFN-γ. Following encapsulation, hydrogels were washed throughout the first 24 hr to simulate sink conditions present in vivo. Hydrogels were then cultured for an additional 3 days after which the hydrogels were degraded, conditioned media collected for cytokine analysis, and hMSCs stained for IDO and PD-L1 followed by flow cytometric analysis (FIG. 3C, 3D). hMSCs encapsulated in hydrogels containing soluble IFN-γ exhibited increased IDO and PD-L1 expression compared to control hMSCs. Importantly, hMSCs encapsulated in hydrogels with tethered cys-IFN-γ showed significantly increased IDO and PD-L1 expression compared to hMSCs encapsulated in hydrogels containing soluble IFN-γ as well as control unstimulated hMSCs. Furthermore, analysis of conditioned media showed that hMSCs encapsulated in cys-IFN-γ-tethered hydrogels secreted increased levels of MCP-1, M-CSF, CXCL9, CXCL10 and CCL8 compared to hMSCs encapsulated in either IFN-γ-containing hydrogels or cells encapsulated in control hydrogels without IFN-γ (FIGS. 3E-3I). In addition, hMSCs encapsulated in cys-IFN-γ-tethered hydrogels had equivalent levels of IL-6, CXCL8, and VEGF as cells encapsulated in IFN-γ-containing hydrogels, and these levels were suppressed compared to control hMSC not exposed to IFN-γ (FIG. 11). Collectively, these results show that cys-IFN-γ-tethered hydrogels significantly alter hMSC phenotype by augmenting the expression and release of immunomodulatory factors.

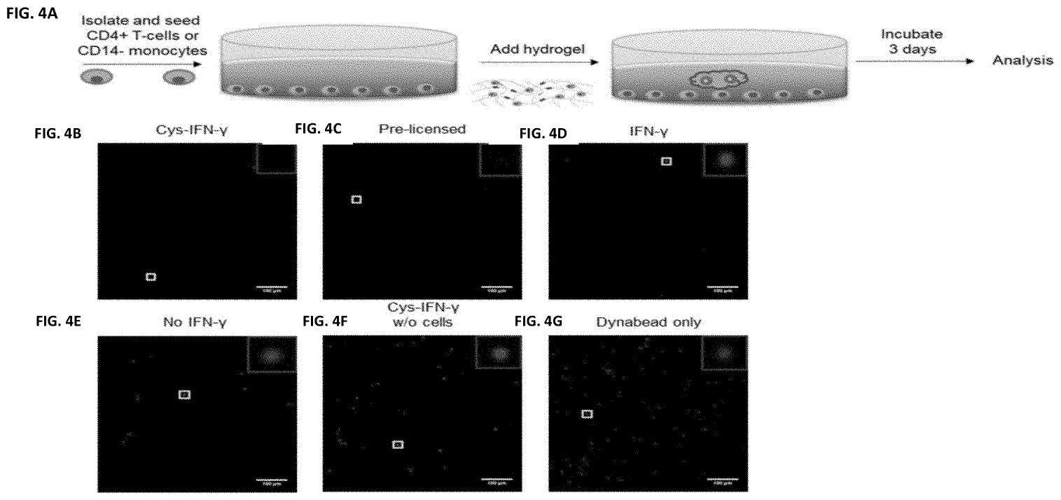

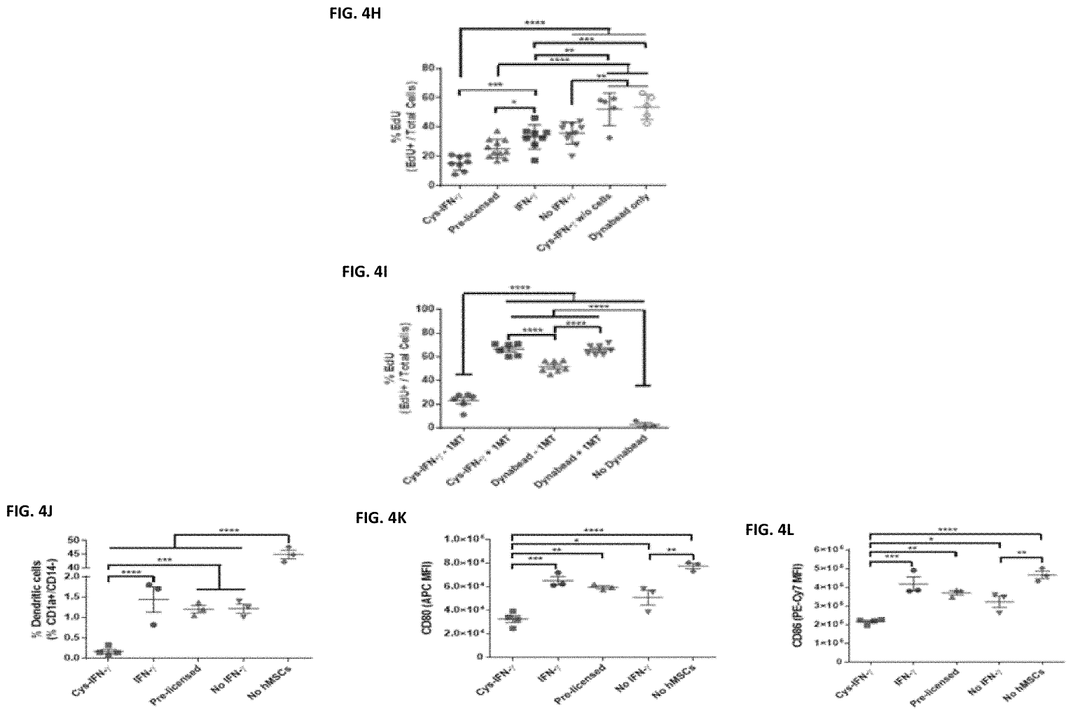

IFN-γ-stimulated hMSCs reduce the proliferation of activated T-cells when co-cultured (Meisel R, et al. Human bone marrow stromal cells inhibit allogeneic T-cell responses by indoleamine 2,3-dioxygenase-mediated tryptophan degradation. Blood. 2004; 103:4619-21). Whether cys-IFN-γ-tethered hydrogels augment the inhibitory effect of hMSCs on T-cell proliferation was therefore studied (FIG. 4A). hMSCs were encapsulated in hydrogels presenting either cys-IFN-γ or IFN-γ and gels with no IFN-γ. Hydrogels were washed twice within 24 hr following encapsulation to simulate a sink effect in vivo. To compare with hMSCs licensed with soluble IFN-γ as routinely done in the literature, a group of hMSCs encapsulated in hydrogels without IFN-γ and incubated in media containing 500 ng/mL IFN-γ (pre-licensed hMSCs) was included. hMSC-laden hydrogels were co-cultured with activated CD4+ human T-cells for 4 days after which the T-cells were stained for EdU and CD3 to examine proliferation and verify their T-cell phenotype, respectively (FIGS. 4B-4G). Activated T-cells cultured solely with Dynabeads (to activate T-cells) showed a similar high degree of proliferation compared to activated T-cells cultured with cys-IFN-γ-tethered hydrogel without hMSCs indicating that the presence of the cys-IFN-γ hydrogel by itself has no effect on T-cell proliferation (FIG. 4H). Furthermore, these two groups showed significantly greater levels of T-cell proliferation compared to all groups having IFN-γ. Importantly, T-cells incubated with hMSCs in cys-IFN-γ-tethered hydrogels exhibited significantly lower levels of proliferation compared to T-cells cultured with hMSCs in hydrogels containing IFN-γ, demonstrating augmented immunomodulatory properties for hMSCs encapsulated in gels with tethered IFN-γ compared to gels with soluble IFN-γ. There were no differences in T-cell proliferation for T-cells incubated with hMSCs in cys-IFN-γ-tethered hydrogels and hydrogels with pre-licensed hMSCs.

The role of IDO produced by hMSCs in this inhibitory effect was then examined. hMSCs were encapsulated in cys-IFN-γ-tethered hydrogels and co-cultured with human T-cells in the presence or absence of the IDO inhibitor, 1-methyl-tryptophan (1-MT) (FIG. 4I). After 4 days, T-cells incubated with hMSCs encapsulated in cys-IFN-γ-tethered hydrogels in the absence of 1-MT exhibited significantly reduced proliferation compared to T-cells cultured in the same conditions in the presence of 1-MT. Importantly, T-cells cultured with 1-MT either with or without hMSCs showed no difference in proliferation. Together, these results show that addition of 1-MT completely inhibited the anti-proliferative effect of licensed hMSCs in the co-culture. This complete abrogation of anti-proliferative effect indicates that IDO is a key regulator of the anti-proliferative activities of hydrogel-encapsulated hMSCs.

In addition to inhibiting T-cell proliferation, IFN-γ-licensed hMSCs inhibit the differentiation of monocytes into dendritic cells in vitro (Spaggiari G M, et al. MSCs inhibit monocyte-derived DC maturation and function by selectively interfering with the generation of immature DCs: central role of MSC-derived prostaglandin E2. Blood. 2009; 113:6576-83). Whether cys-IFN-γ-tethered hydrogels augment the inhibition of dendritic cell differentiation was therefore examined. Untreated or pre-licensed hMSCs were encapsulated within hydrogels containing cys-IFN-γ or IFN-γ and hydrogels containing no IFN-γ and co-cultured with peripheral blood purified CD14+ human monocytes. These cells were co-cultured in dendritic cell differentiation conditions for 5 days. Monocyte differentiation was performed by addition of 100 ng/mL LPS for an additional 2 days. Following complete differentiation, monocytes were stained for the monocyte marker CD14, the dendritic cell marker CD1a and maturation markers CD80 and CD86. Monocytes cultured in the absence of hMSCs exhibited significantly greater dendritic cell differentiation compared to monocytes co-cultured with hMSCs as quantified by the percentage of CD1a+/CD14−cells (FIG. 4J). Monocytes cultured with hydrogels encapsulating untreated hMSCs, hMSCs exposed to soluble IFN-γ (pre-licensed), or hMSCs encapsulated in hydrogels containing IFN-γ showed lower dendritic cell differentiation than monocytes differentiated in the absence of hMSCs, and there were no differences in dendritic cell differentiation among these hMSC-containing groups. Remarkably, monocytes cultured with hMSCs encapsulated in cys-IFN-γ-tethered hydrogels showed a significant reduction in their dendritic cell differentiation compared to monocytes cultured with all other IFN-γ-treated hMSC conditions. Furthermore, monocytes cultured with hMSCs encapsulated in cys-IFN-γ-tethered hydrogels displayed lower expression of maturation markers CD80 and CD86 compared to monocytes cultured in all other conditions tested (FIGS. 4K and 4L). These results show that hMSCs in cys-IFN-γ-tethered hydrogels exhibit significantly upregulated ability to inhibit monocyte-derived dendritic cell differentiation compared to either hMSCs not exposed to IFN-γ or hMSCs in IFN-γ hydrogels.

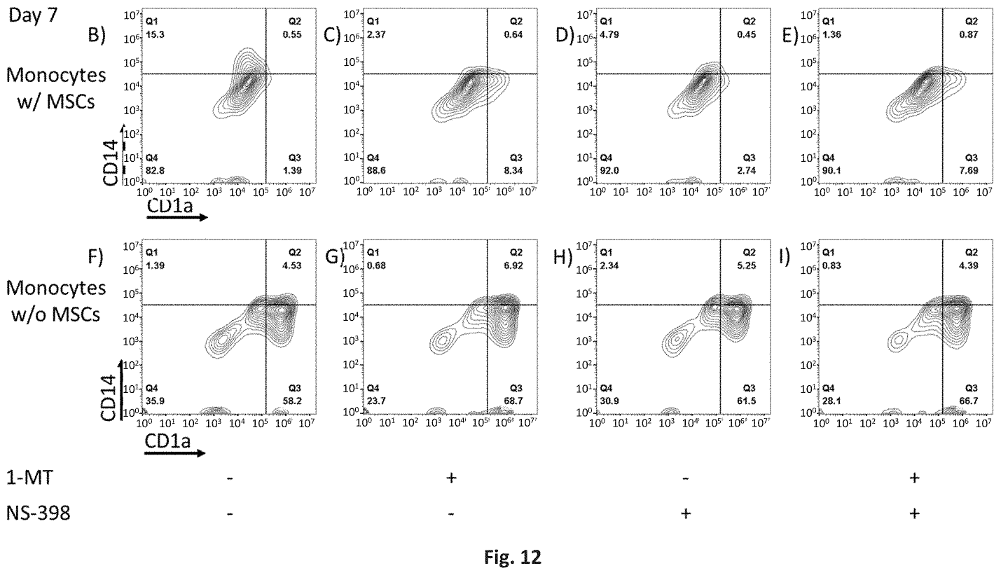

The mechanism of action for this effect was investigated by co-culturing human monocytes with hMSCs encapsulated in cys-IFN-γ-tethered hydrogels in the absence or presence of either an IDO inhibitor (1-MT), a PGE2 inhibitor (NS-398), or both. Following 7 days in dendritic cell differentiation conditions, monocytes were collected, stained for CD1a and CD14 and subjected to flow cytometric analysis (FIG. 12). Without the addition of IDO or PGE2 inhibitor, monocytes co-cultured with hMSCs in cys-IFN-γ-tethered hydrogels showed lower dendritic cell differentiation compared to monocytes that were not co-cultured with hMSCs. Monocytes co-cultured with encapsulated hMSC and exposed to the IDO or PGE2 inhibitor exhibited higher dendritic cell differentiation compared to vehicle only controls. The IDO inhibitor had a more pronounced effect than the PGE2 inhibitor, demonstrating that IDO is the dominant mechanism inhibiting dendritic cell differentiation for hMSCs encapsulated in cys-IFN-γ-tethered hydrogels.

### Example 5

hMSCs in Cys-IFN-γ-Tethered Hydrogels Accelerate Healing of Mucosal Wounds

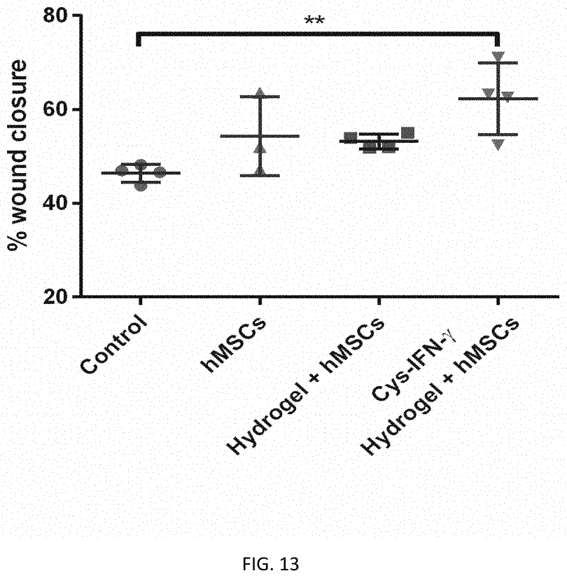

The use of hMSCs for treating inflammatory diseases in clinical trials has rapidly grown in recent years with Crohn's and other inflammatory bowel diseases consisting of a large portion of the conditions being treated (Mao F, et al. Mesenchymal stem cells and their therapeutic applications in inflammatory bowel disease. Oncotarget. 2017; 8:38008-21). In addition, previous literature suggests that licensing hMSCs with IFN-γ can significantly augment the regenerative effects of cell therapy in pre-clinical colitis models (Chen Y, et al. Gene delivery with IFN-gamma-expression plasmids enhances the therapeutic effects of MSCs on DSS-induced mouse colitis. Inflamm Res. 2015; 64:671-81). Whether hMSCs encapsulated within cys-IFN-γ-tethered hydrogels enhance repair of intestinal mucosal wounds was therefore tested. A major advantage of the hydrogel platform is the ability to formulate the scaffold as an injectable delivery vehicle. Because the degradation profile of the hydrogel is an important parameter influencing healing responses, hydrogels crosslinked with a protease-degradable crosslinking peptide (VPM) that previously supported in vivo delivery of therapeutic proteins and excellent tissue healing (Phelps E A, et al. Vasculogenic bio-synthetic hydrogel for enhancement of pancreatic islet engraftment and function in type 1 diabetes. Biomaterials. 2013; 34:4602-11; Shekaran A, et al. Bone regeneration using an alpha 2 beta 1 integrin-specific hydrogel as a BMP-2 delivery vehicle. Biomaterials. 2014; 35:5453-61; Weaver J D, et al. Vasculogenic hydrogel enhances islet survival, engraftment, and function in leading extrahepatic sites. Sci Adv. 2017; 3:e1700184; Johnson C T, et al. Hydrogel delivery of lysostaphin eliminates orthopedic implant infection by Staphylococcus aureus and supports fracture healing. Proc Natl Acad Sci USA. 2018;115:E4960-e9) were used. The effects on wound regeneration of hMSCs delivered within cys-IFN-γ-tethered hydrogels compared to either untreated control wounds, wounds injected with hMSCs in saline, and wounds treated with control hydrogels containing hMSCs were investigated first. Wounds were mechanically induced within the colon of immunocompromised NSG mice using a veterinary colonoscope as described previously (Cruz-Acuna R, et al. Synthetic hydrogels for human intestinal organoid generation and colonic wound repair. Nat Cell Biol. 2017; 19:1326-35). Twenty-four hr following injury, the prescribed treatments were injected at the site of injury and videos of the wounds taken. Five days following treatment, progression of wound repair was recorded and healing was assessed by comparing the wound area on day 5 to the wound area on day 1. Remarkably, wounds treated with hMSCs delivered within cys-IFN-γ-tethered hydrogels enhanced wound healing compared to control untreated wounds (FIG. 13). Importantly, no other groups tested displayed differences compared to the control untreated wounds.

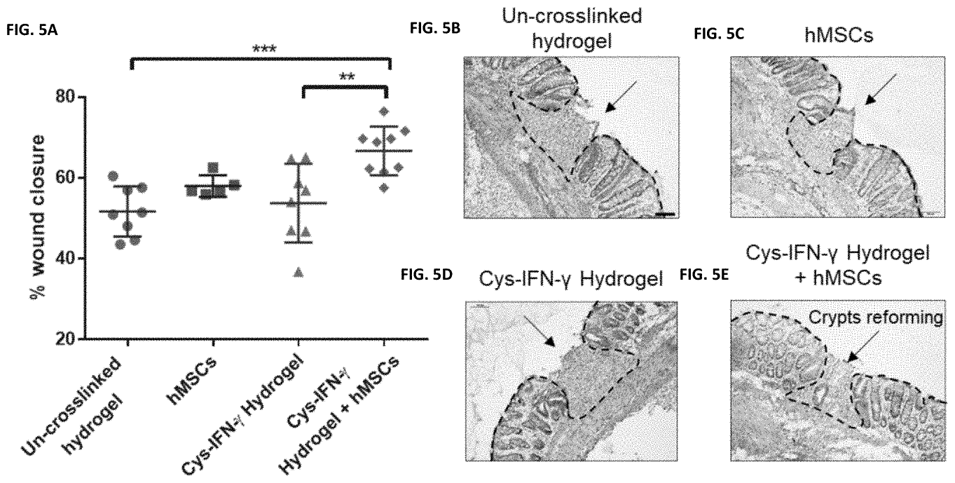

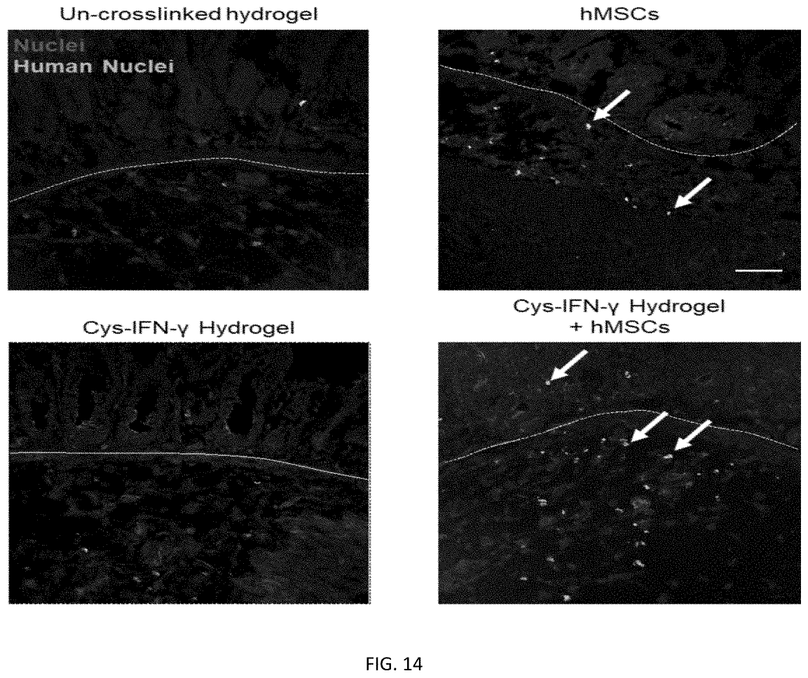

A follow-up experiment was conducted where colonic mucosal wounds in immunocompetent C57/B6 mice were treated with hMSCs delivered within cys-IFN-γ-tethered hydrogels or cys-IFN-γ-tethered hydrogels without hMSCs. The use of C57/B6 mice ensures an active immune system that is more physiologically relevant to clinical cases. Other groups tested included colonic wounds injected with hMSCs in saline and wounds injected with un-crosslinked hydrogel components. Five days post-injury, wound closure was assessed as previously described. No differences in wound closure were noted between mice receiving un-crosslinked hydrogel components and mice injected with either hMSCs in saline or hydrogel-encapsulated hMSCs without cys-IFN-γ. In contrast, hMSCs delivered within cys-IFN-γ-tethered hydrogels exhibited significantly increased wound closure at day 5 post-injury compared to control mice receiving un-crosslinked hydrogel components and mice receiving cys-IFN-γ gels without hMSCs (FIG. 5A). Histological sections confirm this finding showing that mice treated with cys-IFN-γ-tethered hydrogels with hMSCs had smaller wounds compared to the other groups tested (FIG. 5B-E). Notably, wounds treated with cys-IFN-γ-tethered hydrogels with hMSCs showed the presence of crypts re-forming within the repair tissue, indicating healing at a more advanced stage compared other groups. Additionally, wounds examined at 4 weeks post-injection showed the presence of implanted hMSCs, demonstrating persistence of cells that correlates with enhanced wound closure (FIG. 14).

All of the documents cited herein are incorporated herein by reference.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### REFERENCES

[1] Bruder S P, Fink D J, Caplan A I. Mesenchymal stem cells in bone development, bone repair, and skeletal regeneration therapy. J Cell Biochem. 1994; 56:283-94.
[2] Caplan A I. Adult mesenchymal stem cells for tissue engineering versus regenerative medicine. J Cell Physiol. 2007; 213:341-7.
[3] Aggarwal S, Pittenger M F. Human mesenchymal stem cells modulate allogeneic immune cell responses. Blood. 2005; 105:1815-22.
[4] Wang Y, Chen X, Cao W, Shi Y. Plasticity of mesenchymal stem cells in immunomodulation: pathological and therapeutic implications. Nat Immunol. 2014; 15:1009-16.
[5] Gao F, Chiu S M, Motan D A, Zhang Z, Chen L, Ji H L, et al. Mesenchymal stem cells and immunomodulation: current status and future prospects. Cell Death Dis. 2016;7:e2062.
[6] Spaggiari G M, Abdelrazik H, Becchetti F, Moretta L. MSCs inhibit monocyte-derived DC maturation and function by selectively interfering with the generation of immature DCs: central role of MSC-derived prostaglandin E2. Blood. 2009; 113:6576-83.
[7] Meisel R, Zibert A, Laryea M, Gobel U, Daubener W, Dilloo D. Human bone marrow stromal cells inhibit allogeneic T-cell responses by indoleamine 2,3-dioxygenase-mediated tryptophan degradation. Blood. 2004; 103:4619-21.
[8] Spaggiari G M, Capobianco A, Abdelrazik H, Becchetti F, Mingari M C, Moretta L. Mesenchymal stem cells inhibit natural killer-cell proliferation, cytotoxicity, and cytokine production: role of indoleamine 2,3-dioxygenase and prostaglandin E2. Blood. 2008; 111:1327-33.
[9] Corcione A, Benvenuto F, Ferretti E, Giunti D, Cappiello V, Cazzanti F, et al. Human mesenchymal stem cells modulate B-cell functions. Blood. 2006; 107:367-72.
[10] Auletta J J, Eid S K, Wuttisarnwattana P, Silva I, Metheny L, Keller M D, et al. Human mesenchymal stromal cells attenuate graft-versus-host disease and maintain graft-versus-leukemia activity following experimental allogeneic bone marrow transplantation. Stem Cells. 2015; 33:601-14.
[11] Wang X, Kimbrel E A, Ijichi K, Paul D, Lazorchak A S, Chu J, et al. Human ESC-derived MSCs outperform bone marrow MSCs in the treatment of an EAE model of multiple sclerosis. Stem Cell Reports. 2014; 3:115-30.
[12] Gonzalez M A, Gonzalez-Rey E, Rico L, Buscher D, Delgado M. Adipose-derived mesenchymal stem cells alleviate experimental colitis by inhibiting inflammatory and autoimmune responses. Gastroenterology. 2009; 136:978-89.
[13] Yanez R, Lamana M L, Garcia-Castro J, Colmenero I, Ramirez M, Bueren J A. Adipose tissue-derived mesenchymal stem cells have in vivo immunosuppressive properties applicable for the control of the graft-versus-host disease. Stem Cells. 2006; 24:2582-91.
[14] Forbes G M, Sturm M J, Leong R W, Sparrow M P, Segarajasingam D, Cummins A G, et al. A phase 2 study of allogeneic mesenchymal stromal cells for luminal Crohn's disease refractory to biologic therapy. Clin Gastroenterol Hepatol. 2014; 12:64-71.
[15] Le Blanc K, Frassoni F, Ball L, Locatelli F, Roelofs H, Lewis I, et al. Mesenchymal stem cells for treatment of steroid-resistant, severe, acute graft-versus-host disease: a phase II study. Lancet. 2008; 371:1579-86.
[16] Resnick I B, Barkats C, Shapira M Y, Stepensky P, Bloom A I, Shimoni A, et al. Treatment of severe steroid resistant acute GVHD with mesenchymal stromal cells (MSC). Am J Blood Res. 2013; 3:225-38.
[17] Panes J, Garcia-Olmo D, Van Assche G, Colombel J F, Reinisch W, Baumgart D C, et al. Expanded allogeneic adipose-derived mesenchymal stem cells (Cx601) for complex perianal fistulas in Crohn's disease: a phase 3 randomised, double-blind controlled trial. Lancet. 2016; 388:1281-90.
[18] Krampera M, Cosmi L, Angeli R, Pasini A, Liotta F, Andreini A, et al. Role for interferon-gamma in the immunomodulatory activity of human bone marrow mesenchymal stem cells. Stem Cells. 2006; 24:386-98.
[19] Lee M W, Ryu S, Kim D S, Sung K W, Koo H H, Yoo K H. Strategies to improve the immunosuppressive properties of human mesenchymal stem cells. Stem Cell Res Ther. 2015; 6:179.
[20] Polchert D, Sobinsky J, Douglas G, Kidd M, Moadsiri A, Reina E, et al. IFN-gamma activation of mesenchymal stem cells for treatment and prevention of graft versus host disease. Eur J Immunol. 2008; 38:1745-55.
[21] Liang C, Jiang E, Yao J, Wang M, Chen S, Zhou Z, et al. Interferon-gamma mediates the immunosuppression of bone marrow mesenchymal stem cells on T-lymphocytes in vitro. Hematology. 2018; 23:44-9.
[22] Jin P, Zhao Y, Liu H, Chen J, Ren J, Jin J, et al. Interferon-gamma and tumor necrosis factor-alpha polarize bone marrow stromal cells uniformly to a Th1 phenotype. Sci Rep. 2016; 6:26345.
[23] Bernardo M E, Fibbe W E. Mesenchymal stromal cells: sensors and switchers of inflammation. Cell Stem Cell. 2013; 13:392-402.
[24] Shi Y, Su J, Roberts A I, Shou P, Rabson A B, Ren G. How mesenchymal stem cells interact with tissue immune responses. Trends Immunol. 2012; 33:136-43.
[25] Chinnadurai R, Copland I B, Patel S R, Galipeau J. IDO-independent suppression of T cell effector function by IFN-gamma-licensed human mesenchymal stromal cells. J Immunol. 2014; 192:1491-501.
[26] Duijvestein M, Wildenberg M E, Welling M M, Hennink S, Molendijk I, van Zuylen V L, et al. Pretreatment with interferon-gamma enhances the therapeutic activity of mesenchymal stromal cells in animal models of colitis. Stem Cells. 2011; 29:1549-58.
[27] Heathman T R, Nienow A W, McCall M J, Coopman K, Kara B, Hewitt C J. The translation of cell-based therapies: clinical landscape and manufacturing challenges. Regen Med. 2015; 10:49-64.
[28] Tarnowski J, Krishna D, Jespers L, Ketkar A, Haddock R, Imrie J, et al. Delivering advanced therapies: the big pharma approach. Gene Ther. 2017; 24:593-8.
[29] Dodson B P, Levine A D. Challenges in the translation and commercialization of cell therapies. BMC Biotechnol. 2015; 15:70.
[30] Veith A P, Henderson K, Spencer A, Sligar A D, Baker A B. Therapeutic strategies for enhancing angiogenesis in wound healing. Adv Drug Deliv Rev. 2018; 50169-409X(18)30246-1.
[31] Chuang C K, Lin K J, Lin C Y, Chang Y H, Yen T C, Hwang S M, et al. Xenotransplantation of human mesenchymal stem cells into immunocompetent rats for calvarial bone repair. Tissue Eng Part A. 2010; 16:479-88.
[32] Lee K, Silva E A, Mooney D J. Growth factor delivery-based tissue engineering: general approaches and a review of recent developments. J R Soc Interface. 2011; 8:153-70.
[33] Annabi N, Tamayol A, Uquillas J A, Akbari M, Bertassoni L E, Cha C, et al. 25th anniversary article: Rational design and applications of hydrogels in regenerative medicine. Adv Mater. 2014; 26:85-123.
[34] Dominici M, Le Blanc K, Mueller I, Slaper-Cortenbach I, Marini F, Krause D, et al. Minimal criteria for defining multipotent mesenchymal stromal cells. The International Society for Cellular Therapy position statement. Cytotherapy. 2006; 8:315-7.
[35] Fam C M, Eisenberg S P, Carlson S J, Chlipala E A, Cox G N, Rosendahl M S. PEGylation improves the pharmacokinetic properties and ability of interferon gamma to inhibit growth of a human tumor xenograft in athymic mice. J Interferon Cytokine Res. 2014; 34:759-68.
[36] Klinker M W, Marklein R A, Lo Surdo J L, Wei C H, Bauer S R. Morphological features of IFN-gamma-stimulated mesenchymal stromal cells predict overall immunosuppressive capacity. Proc Natl Acad Sci USA. 2017;114:E2598-e607.
[37] Grohmann U, Fallarino F, Puccetti P. Tolerance, DCs and tryptophan: much ado about IDO. Trends Immunol. 2003; 24:242-8.
[38] Zhang Q, Shi S, Liu Y, Uyanne J, Shi Y, Shi S, et al. Mesenchymal stem cells derived from human gingiva are capable of immunomodulatory functions and ameliorate inflammation-related tissue destruction in experimental colitis. J Immunol. 2009; 183:7787-98.
[39] Cruz-Acuna R, Quiros M, Farkas A E, Dedhia P H, Huang S, Siuda D, et al. Synthetic hydrogels for human intestinal organoid generation and colonic wound repair. Nat Cell Biol. 2017; 19:1326-35.
[40] Garcia J R, Clark A Y, Garcia A J. Integrin-specific hydrogels functionalized with VEGF for vascularization and bone regeneration of critical-size bone defects. J Biomed Mater Res A. 2016; 104:889-900.
[41] Stevens M M, George J H. Exploring and engineering the cell surface interface. Science. 2005; 310:1135-8.
[42] Mao F, Tu Q, Wang L, Chu F, Li X, Li H S, et al. Mesenchymal stem cells and their therapeutic applications in inflammatory bowel disease. Oncotarget. 2017; 8:38008-21.
[43] Chen Y, Song Y, Miao H, Xu Y, Lv M, Wang T, et al. Gene delivery with IFN-gamma-expression plasmids enhances the therapeutic effects of MSCs on DSS-induced mouse colitis. Inflamm Res. 2015; 64:671-81.
[44] Phelps E A, Headen D M, Taylor W R, Thule P M, Garcia A J. Vasculogenic bio-synthetic hydrogel for enhancement of pancreatic islet engraftment and function in type 1 diabetes. Biomaterials. 2013; 34:4602-11.
[45] Shekaran A, Garcia J R, Clark A Y, Kavanaugh T E, Lin A S, Guldberg R E, et al. Bone regeneration using an alpha 2 beta 1 integrin-specific hydrogel as a BMP-2 delivery vehicle. Biomaterials. 2014; 35:5453-61.
[46] Weaver J D, Headen D M, Aquart J, Johnson C T, Shea L D, Shirwan H, et al. Vasculogenic hydrogel enhances islet survival, engraftment, and function in leading extrahepatic sites. Sci Adv. 2017;3:e1700184.
[47] Johnson C T, Wroe J A, Agarwal R, Martin K E, Guldberg R E, Donlan R M, et al. Hydrogel delivery of lysostaphin eliminates orthopedic implant infection by Staphylococcus aureus and supports fracture healing. Proc Natl Acad Sci USA. 2018;115:E4960-e9.
[48] Squillaro T, Peluso G, Galderisi U. Clinical Trials With Mesenchymal Stem Cells: An Update. Cell Transplant. 2016; 25:829-48.
[49] Klinker M W, Wei C H. Mesenchymal stem cells in the treatment of inflammatory and autoimmune diseases in experimental animal models. World J Stem Cells. 2015; 7:556-67.
[50] Wang L T, Ting C H, Yen M L, Liu K J, Sytwu H K, Wu K K, et al. Human mesenchymal stem cells (MSCs) for treatment towards immune- and inflammation-mediated diseases: review of current clinical trials. J Biomed Sci. 2016; 23:76.
[51] Krampera M. Mesenchymal stromal cell ‘licensing’: a multistep process. Leukemia. 2011; 25:1408-14.
[52] Cosgrove B D, Mui K L, Driscoll T P, Caliari S R, Mehta K D, Assoian R K, et al. N-cadherin adhesive interactions modulate matrix mechanosensing and fate commitment of mesenchymal stem cells. Nat Mater. 2016; 15:1297-306.
[53] Li H, Koenig A M, Sloan P, Leipzig N D. In vivo assessment of guided neural stem cell differentiation in growth factor immobilized chitosan-based hydrogel scaffolds. Biomaterials. 2014; 35:9049-57.
[54] Li R, Xu J, Wong D S H, Li J, Zhao P, Bian L. Self-assembled N-cadherin mimetic peptide hydrogels promote the chondrogenesis of mesenchymal stem cells through inhibition of canonical Wnt/beta-catenin signaling. Biomaterials. 2017; 145:33-43.
[55] Zhang K, Jia Z, Yang B, Feng Q, Xu X, Yuan W, et al. Adaptable hydrogels mediate cofactor-assisted activation of biomarker-responsive drug delivery via positive feedback for enhanced tissue regeneration. Adv Sci. 2018; 5:1800875.

1. A composition comprising a scaffold, a cell, and a licensing agent, wherein the licensing agent is covalently attached to the scaffold, and the cell is non-covalently attached to the scaffold.

2. The composition of claim 1, further comprising at least one linker A, wherein linker A is covalently attached to the scaffold.

3. The composition of claim 2, wherein linker A is a peptide linker.

4. (canceled)

5. The composition of claim 3, wherein the peptide linker comprises a cell attachment amino acid sequence.

6. (canceled)

7. The composition of claim 5, wherein a cell is non-covalently attached to the cell attachment amino acid sequence in linker A.

8. The composition of claim 1, further comprising at least one linker B capable of covalently joining two or more scaffolds together.

9. The composition of claim 8, wherein linker B is a peptide linker.

10.-11. (canceled)

12. The composition of claim 9, wherein the peptide linker comprises at least two cysteine residues.

13. The composition of claim 12, wherein the peptide linker is covalently joined to the scaffold through at least one cysteine residue in linker B.

14. The composition of claim 1, wherein the scaffold comprises at least one cysteine-reactive moiety.

15. The composition of claim 14, wherein the at least one cysteine-reactive moiety is a maleimide group.

16. The composition of claim 1, wherein the licensing agent is selected from the group consisting of a protein, a cytokine, a nucleic acid, a hormone, a polysaccharide, and a lipid.

17.-19. (canceled)

20. The composition of claim 1, wherein the scaffold is a polyethylene glycol.

21.-24. (canceled)

25. The composition of claim 1, wherein the cell is selected from the group consisting of a mesenchymal stem cell, an induced pluripotent stem cell, and an embryonic stem cell.

26.-27. (canceled)

28. The composition of claim 16, wherein the protein is selected from the group consisting of interferon gamma, interleukin-1 alpha, interleukin-1 beta, and tumor necrosis factor.

29.-36. (canceled)

37. The composition of claim 1, wherein the cell is a mesenchymal stem cell and wherein the licensing agent is an interferon gamma cysteine variant.

38.-45. (canceled)

46. A method for stimulating tissue regeneration in an animal, comprising administering the composition of claim 1 to at least one damaged tissue in an animal.

47.-50. (canceled)

51. A method for treating a disease in an animal, comprising administering the composition of claim 1 to an animal with a disease treatable with the composition.

52.-59. (canceled)

60. A composition comprising a scaffold, a cell, and a licensing agent, wherein the licensing agent is covalently attached to the scaffold, and the cell is encapsulated within the composition.

61. The composition of claim 60, further comprising at least one linker B wherein the at least one linker B is covalently attached to the scaffold.

62.-65. (canceled)

66. A composition comprising a scaffold, a licensing agent, and at least one linker B, wherein the licensing agent is covalently attached to the scaffold, and the at least one linker B is covalently attached to the scaffold.

67.-69. (canceled)

70. The composition of claim 1, further comprising at least one linker A and at least one linker B, wherein the linker A and the linker B are the same.
